(19) United States
(12) Patent Application Publication
(10) Pub. No.: US 2011/0081392 A1
(43) Pub. Date: Apr. 7, 2011

(54) Shampoo Compositions

(76) Inventors: Renato de Arruda, Campinas-SP (BR); Clementine Calvet, Gosselies (BE); Anick Colson, Vedrin (BE); Alice Devinat, Montbeliard (FR)

(21) Appl. No.: 12/996,396

(22) PCT Filed: Jun. 18, 2009
(86) PCT No.: PCT/EP2009/057604

§ 371 Date: Dec. 6, 2010

(30) Foreign Application Priority Data
Jun. 20, 2008 (GB) 0811302.9

(51) Int. Cl. A61K 8/02; A61K 8/60; A61K 8/89; A61Q 5/12; A61Q 5/02; A61K 8/23; A61K 8/73; A61K 8/19; B05D 5/00

(52) U.S. Cl. 424/401; 510/119; 424/70.12; 510/160; 510/130; 427/212

(57) ABSTRACT

The invention relates to a granulated personal care shampoo comprising a shampoo composition comprising at least one surfactant agglomerated with a water-soluble, water-dispersible or water-insoluble solid particulate carrier. Preferably, the shampoo composition additionally contains a conditioner preferably comprising an organopolysiloxane. The granulated shampoo of the invention can dissolve readily with formation of shampoo foam in hot or cold water, and can be perceived as soft in the dry state, free flowing state and providing a pleasant feel on the skin. The granulated shampoo can be packaged in various types of biodegradable packaging such as paper (environmentally more friendly than plastic sachets) to form a stable package which do not deteriorate on storage.

This invention relates to shampoo. By shampoo is generally meant a cleaning personal care product which is designed to clean skin or hair. In the present description, we use the term “shampoo” to include shampoos for animals as well as for human hair, and also to include body shampoo and other personal care products. The personal care product may be functional with respect to the portion of the body to which it is applied; it can be cosmetic, therapeutic, or some combination thereof. For example it can be chosen from: personal or facial cleansers, bath powders, shaving soaps, shaving lathers, hair conditioners, oil removers and colour cosmetic removers. A body shampoo can be for example a body wash or shower cleaner also called “shower gel”. A hair shampoo is designed to remove oils, dirt, skin particles, dandruff, environmental pollutants and other contaminant particles that gradually build up in hair, without eliminating all surface lipids as sebum. Sebum is a natural protecting layer which is composed of triglycerides, free fatty acids, waxes, cholesterol esters, squalenes and paraffins. The invention is concerned in particular with shampoo in the form of a free flowing powder able to clean hair or skin when wetted with water.

Shampoos are usually sold in liquid format, most commonly in bottles containing enough shampoo for several hair washes. There is however a requirement for single dose packages of shampoo, particularly in countries where the cost of a multi-dose bottle of shampoo is a major expense. Single dose packages of shampoo have generally been sold in plastic sachets, which are not recycled or biodegradable. A shampoo in stable powder form could be packaged in a more environmentally friendly material.

U.S. Pat. No. 4,330,438 describes a powder shampoo concentrate comprising a mixture of an anionic surfactant and a nonionic derivative of a polygalactomannan gum together with conventional shampoo ingredients. U.S. Pat. No. 6,451,297 describes a hair care product in the form of a powder having a granulometry of 30 to 500 microns, applicable directly to the wet hair and/or the body and comprising less than 40% of at least one surfactant, and from 1 to 12% of at least one perfume, the percentage being made up to 100% by one or more products selected from the group consisting of sugars, starches, celluloses, polyols, proteins, amino acids, perfumes, colourings, antioxidants, plant substances, seaweed, vitamins, essential oils and mineral fillers. Such powder shampoos are prepared by blending powder raw materials in powders. This approach forms powders which do not dissolve readily enough and tend to give some grains upon dissolution. Furthermore, only solid raw materials can be incorporated.

DE4214480 describes a dried powdered shampoo which is applied to wet hair where it combines with water to form a normal shampoo, with the advantage of cost reduction in the plastic packaging required for containers.

US2004/0202632 describes foamed solid cosmetic compositions which are prepared by warming fatty or oil-based materials to 70[deg.] C. to achieve fluidity. Other liquid or solid non-meltable materials are then dispersed into the resultant mass with thorough mixing. The product resulting therefrom is then added with mixing to a high amylose destructurized corn starch. This formed mass is then extruded at a temperature of 150-250[deg.] C. The extruded mass is then shaped. Fragrance is sprayed onto the shaped mass. In this manner a shampoo solid is prepared. Destructurized starch is water dissolvable. It is generated under high of temperature, pressure, shear, limited water and sufficient time. For instance, natural starch can be treated at elevated temperature in a closed vessel.

EP1908493 discloses pulverized, non-fluid hair conditioning products made by first dissolving a gas in said fluid hair conditioning composition at high pressure, then expanding the liquid/gas solution, wherein said solid carrier is added either before, or during or shortly after said expansion. The products can be used in a method of conditioning human hair.

U.S. Pat. No. 4,035,267 describes a dry shampoo containing chitin powder. WO 2003/049711 describes the use of a siliconized elastomeric complex for making a dry aerosol shampoo sprayed with at least a hydrocarbon propellant. These shampoos are intended to be used dry and are removed from the hair by brushing. The efficiency of dry shampoos to clean hair is much lower than liquid shampoos applied with water.

A shampoo product according to the present invention is a granulated personal care shampoo comprising a shampoo composition, comprising at least one surfactant, agglomerated onto solid carrier particles. By granules we mean agglomerated particles preferably free flowing particles as opposed to slurry agglomerate. Granules according to the invention are preferably granules containing carrier particles upon which a shampoo composition is deposited.

In a process according to the invention for the preparation of a personal care shampoo, a liquid shampoo composition comprising at least one surfactant which has been molten, dispersed or solubilised in a liquid is contacted with a solid particulate carrier under conditions such that the surfactant is agglomerated with the carrier, the agglomerated product being kept in granule form during agglomeration or subsequently formed into granules. The process does not comprise extrusion techniques.

We have found that the granulated shampoo of the invention can dissolve readily with formation of shampoo foam in hot or cold water, and can be perceived as soft in the dry state, free flowing state and providing a pleasant feel on the skin. The granulated shampoo can be packaged in various types of biodegradable packaging such as paper (environmentally more friendly than plastic sachets) to form a stable package which do not deteriorate on storage.

The granulated shampoo according to the invention needs to be put in presence of water to become effective. It is not intended to be used as a dry shampoo.

The surfactant used in the granulated personal care shampoo can be any of those known for use in personal care products and can be selected from anionic, cationic, nonionic and amphoteric surfactants. More than one surfactant can be used, for example different types of surfactants or more than one surfactant of the same type (ionic or nonionic).

Examples of suitable anionic surfactants include alkali metal sulforicinates, sulfonated glyceryl esters of fatty acids such as sulfonated monoglycerides of coconut oil acids, salts of sulfonated monovalent alcohol esters such as sodium oleylisethianate, metal soaps of fatty acids, amides of amino sulfonic acids such as the sodium salt of oleyl methyl tauride, sulfonated products of fatty acids nitriles such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanolamine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, alkylarylsulfonates having 1 or more alkyl groups of 8 or more carbon atoms, alkylbenzenesulfonic acid alkali metal salts exemplified by hexylbenzenesulfonic acid sodium salt, octylbenzenesulfonic acid sodium salt, decylbenzenesulfonic acid sodium salt, dodecylbenzenesulfonic acid sodium salt, cetylbenzenesulfonic acid sodium salt, and myristylbenzenesulfonic acid sodium salt, sulphuric esters of polyoxyethylene alkyl ether including CH₃(CH₂)₆CH₂O(C₂H₄O)₂SO₃H, CH₃(CH₂)₇CH₂O(C₂H₄O)_{3.5}SO₃H, CH₃(CH₂)₈CH₂O(C₂H₄O)₈SO₃H, CH₃(CH₂)₁₉CH₂O(C₂H₄O)₄SO₃H, and CH₃(CH₂)₁₀CH₂O(C₂H₄O)₆SO₃H, sodium salts, potassium salts, and amine salts of alkylnapthylsulfonic acid.

Preferably the detersive surfactant is selected from the group consisting of sodium lauryl sulfate, ammonium lauryl sulfate, triethanolamine lauryl sulfate, sodium lauryl ether sulfate, and ammonium lauryl ether sulfate, alkali metal salts of dialkyl sulphosuccinates available from American Cyanamid Company, Wayne, N.J. under the general tradename Aerosol. The anionic detersive surfactant is present in the shampoo compositions of this invention in an amount from about 1 to 50 wt % and preferably about 5 to 25 wt % based on the total weight of the dry composition.

Examples of cationic surfactants include various fatty acid amines and amides and their derivatives, and the salts of the fatty acid amines and amides. Examples of aliphatic fatty acid amines include dodecylamine acetate, octadecylamine acetate, and acetates of the amines of tallow fatty acids, homologues of aromatic amines having fatty acids such as dodecylanalin, fatty amides derived from aliphatic diamines such as undecylimidazoline, fatty amides derived from aliphatic diamines such as undecylimidazoline, fatty amides derived from disubstituted amines such as oleylaminodiethylamine, derivatives of ethylene diamine, quaternary ammonium compounds and their salts which are exemplified by tallow trimethyl ammonium chloride, dioctadecyldimethyl ammonium chloride, didodecyldimethyl ammonium chloride, dihexadecyl ammonium chloride, alkyltrimethylammonium hydroxides such as octyltrimethylammonium hydroxide, dodecyltrimethylammonium hydroxide, or hexadecyltrimethylammonium hydroxide, dialkyldimethylammonium hydroxides such as octyldimethylammonium hydroxide, decyldimethylammonium hydroxide, didodecyldimethylammonium hydroxide, dioctadecyldimethylammonium hydroxide, tallow trimethylammonium hydroxide, trimethylammonium hydroxide, methylpolyoxyethylene cocoammonium chloride, and dipalmityl hydroxyethylammonium methosulfate, amide derivatives of amino alcohols such as beta-hydroxylethylstearylamide, and amine salts of long chain fatty acids.

Examples of suitable cationic surfactants include also quaternary ammonium halides such as octyl trimethyl ammonium chloride, dodecyl trimethyl ammonium chloride, hexadecyl trimethyl ammonium chloride, octyl dimethyl benzyl ammonium chloride, decyl dimethyl benzyl ammonium chloride and coco trimethyl ammonium chloride as well as other salts of these materials, fatty amines and basic pyridinium compounds, quaternary ammonium bases of benzimidazolines, polypropanolpolyethanol amines, polyethoxylated quaternary ammonium salts and ethylene oxide condensation products of the primary fatty amines, available from Armak Company, Chicago, Ill. under the tradenames Ethoquad, Ethomeen, or Arquad. It can also be an esterquat type compound. A preferred type of quaternary ammonium material are those derived from triethanolamine (hereinafter referred to as ‘TEA quats’) as described in for example U.S. Pat. No. 3,915,867 and represented by formula: (TOCH₂CH₂)₃N+(R₉) wherein T is H or (R₈—CO—) where R₈ group is independently selected from C₈₋₂₈ alkyl or alkenyl groups and R₉ is C₁₋₄alkyl or hydroxyalkyl groups or C₂₋₄ alkenyl groups. For example N-methyl-N,N,N-triethanolamine ditallowester or di-hardened-tallowester quaternary ammonium chloride or methosulphate. Examples of commercially available TEA quats include Rewoquat WE18 and Rewoquat WE20, both partially unsaturated (ex. WITCO), Tetranyl AOT-1, fully saturated (ex. KAO) and Stepantex VP 85, fully saturated (ex. Stepan).

Examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters.

Suitable nonionic surfactants include condensates of ethylene oxide with a long chain (fatty) alcohol or (fatty) acid, condensates of ethylene oxide with an amine or an amide, condensation products of ethylene and propylene oxides, fatty acid alkylol amide and fatty amine oxides. Examples of non-ionic surfactants include polyoxyalkylene alkyl ethers such as polyethylene glycol long chain (12-14C) alkyl ether, polyoxyalkylene sorbitan ethers, polyoxyalkylene alkoxylate esters, polyoxyalkylene alkylphenol ethers, ethylene glycol propylene glycol copolymers, polyvinyl alcohol and alkylpolysaccharides.

Preferred surfactants include trimethylnonyl polyethylene glycol ethers and polyethylene glycol ether alcohols containing linear alkyl groups having from 11 to 15 such as 2,6,8-trimethyl-4-nonyloxypolyethylene oxyethanol (6 EO) (sold as Tergitol® TMN-6 by OSi Specialties, A Witco Company, Endicott, N.Y.), 2,6,8-trimethyl-4-nonyloxypolyethylene oxyethanol (10 EO) (sold as Tergitol® TMN-10 by OSi Specialties, A Witco Company, Endicott, N.Y.), alkylene-oxypolyethylene oxyethanol (C₁₁₋₁₅ secondary alkyl, 9 EO) (sold as Tergitol® 15-S-9 by OSi Specialties, A Witco Company, Endicott, N.Y.), alkylene-oxypolyethylene oxyethanol (C₁₁₋₁₅ secondary alkyl, 15 EO) (sold as Tergitol® 15-S-15 by OSi Specialties, A Witco Company, Endicott, N.Y.), nonionic ethoxylated tridecyl ethers available from Emery Industries, Mauldin, S.C. under the general tradename Trycol.

The amphoteric surfactants, whose nature is not a critical feature in the context of the present invention, can be, in particular (non-limiting list), aliphatic secondary or tertiary amine derivatives in which the aliphatic radical is a linear or branched chain containing 8 to 22 carbon atoms and containing-at least one water-soluble anionic group (for example carboxylate, sulphonate, sulphate, phosphate or phosphonate); mention may also be made of (C₈-C₂₀)alkyl-betaines, sulphobetaines, (C₈-C₂₀)alkylamido(C₁-C₆)alkyl-betaines or (C₈-C₂₀)alkylamido(C₁-C₆)alkylsulphobetaines.

Among the amine derivatives, mention may be made of the products sold under the name MIRANOL®, as described in U.S. Pat. Nos. 2,528,378 and 2,781,354 and with the structures:

R₂—CONHCH₂CH₂—N(R₃)(R₄)(CH₂COO—) (2)

in which: R₂denotes an alkyl radical derived from an acid R₂—COOH present in hydrolysed coconut oil, a heptyl, nonyl or undecyl radical, R₃ denotes a β-hydroxyethyl group and R₄ a carboxymethyl group; and

R_{2-CONHCH2}CH₂—N(B)(C) (3)

in which:
B represents—CH₂CH₂OX′, C represents—(CH₂)_{z}1'Y′, with z=1 or 2,
X′ denotes the—CH₂CH₂—COOH group or a hydrogen atom,
Y′ denotes—COOH or the—CH₂—CHOH—SO₃H radical,
R_{2′} denotes an alkyl radical of an acid R₉—COOH present in coconut oil or in hydrolysed linseed oil, an alkyl radical, in particular a C₇, C₉, C₁₁or C₁₃ alkyl radical, a C₁₇alky radical and its iso form, or an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, and Cocoamphodipropionic acid. By way of example, mention may be made of the Cocoamphodiacetate sold under the trade name MIRANOL® C2M concentrate by the company Rhodia Chimie.

In the compositions in accordance with the invention, mixtures of surfactants and in particular mixtures of anionic surfactants and of amphoteric or nonionic surfactants are preferably used. One mixture which is particularly preferred is a mixture consisting of at least one anionic surfactant and at least one amphoteric surfactant.

Preferably, the shampoo composition has a pH when wetted with water of around neutral, for example pH 4.0 to 9.5, more preferably 4.5 to 8.5, even more preferably 7 to 8.5, to avoid irritating the skin. Preferably the surfactant or blend of surfactants used generates a neutral pH on mixing with water. If anionic surfactants are used, it may be preferred that an anionic surfactant is used in conjunction with a cationic or amphoteric surfactant, and similarly a cationic surfactant may be used in conjunction with an anionic or amphoteric surfactant.

Before contacting the carrier, the surfactant, which is usually a blend of surfactants, is made liquid to form a liquid shampoo composition. When starting from solid surfactant, it can be molten to obtain a liquid shampoo composition, which may be further diluted with water if necessary. A water-soluble liquid surfactant can be diluted in water. A particulate surfactant can be dispersed in water. Preferably, the surfactant is mixed with sufficient water to be wetted. More preferably the surfactant is mixed with sufficient water to dissolve any solid surfactant.

The shampoo composition may additionally include a water-soluble or water-dispersible binder to improve the stability of the granules. Some of the surfactants or foam boosters can act as binders to some extent but a further binder can be added to provide extra handling stability if required. Examples of binders are polycarboxylates, for example polyacrylic acid or a partial sodium salt thereof or a copolymer of acrylic acid, for example a copolymer with maleic anhydride, polyoxyalkylene polymers such as polyethylene glycol, which can be applied molten or as an aqueous solution, reaction products of tallow alcohol and ethylene oxide, or cellulose ethers, particularly water-soluble or water-swellable cellulose ethers such as sodium carboxymethylcellulose, or sugar syrup binders such as Polysorb 70/12/12 or LYCASIN 80/55 HDS maltitol syrup or Roclys C1967 S maltodextrin solution.

Polycarboxylate materials are water soluble polymers, copolymers or salts thereof. They have at least 60% by weight of segments with the general formula:

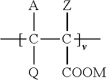

wherein A, Q and Z are each selected from the group consisting of hydrogen, methyl, carboxy, carboxymethyl, hydroxy and hydroxymethyl, M is hydrogen, alkali metal, ammonium or substituted ammonium and v is from 30 to 400. Preferably A is hydrogen or hydroxy, Q is hydrogen or carboxy and Z is hydrogen. Suitable polymeric polycarboxylates include polymerised products of unsaturated monomeric acids, e.g. acrylic acid, maleic acid, maleic anhydride, fumaric acid, itaconic acid, aconitic acid, mesaconic acid, citraconic acid and methylenemalonic acid. The copolymerisation with lesser amounts of monomeric materials comprising no carboxylic acid, e.g. vinylmethyl, vinylmethylethers, styrene and ethylene is not detrimental to the use of the polycarboxylates of the present invention. Depending on the type of polycarboxylate this level can be kept low, or levels can be up to about 40% by weight of the total polymer or copolymer.

Particularly suitable polymeric polycarboxylates are polyacrylates with an average viscosity at 25° C. in mPa·s from 50 to 10,000, preferably 2,000 to 8,000. The most preferred polycarboxylate polymers are acrylate/maleate or acrylate/fumarate copolymers or their sodium salts. Molar mass of suitable polycarboxylates may be in the range from 1,000 to 500,000, preferably 3,000 to 100,000, most preferably 15,000 to 80,000. The ratio of acrylate to maleate or fumarate segments is preferably in the range from 30:1 to 2:1.

The water-soluble or water-dispersible binder can be mixed with the liquid shampoo composition before being deposited on the carrier, or alternatively is separately deposited on the carrier particles either at the same time or subsequently, or at both times. In both cases, the binder should be liquid, being solubilised or molten. The binder component can for example be used at 0.1 to 10% by weight of the dry shampoo composition.

In some embodiments, the liquid shampoo composition contains at least 1, preferably at least 1.5% by weight water, and preferably the liquid shampoo composition contains at least 20% by weight water. In other embodiments, liquid shampoo compositions containing up to 75% water can be used.

The solid particulate carrier is preferably water-soluble or water-dispersible. Examples of water soluble carriers include water soluble salts such as sodium sulfate, sodium acetate, sodium silicate, magnesium sulfate, phosphates, for example powdered or granular sodium tripolyphosphate, sodium bicarbonate, sodium perborate, sodium citrate and water soluble carbohydrates such as cellulose derivatives, for example sodium carboxymethylcellulose, or sugars, for example lactose, dextrose, or maltodextrin, for example that sold under the Trade Mark ‘Glucidex IT’. Examples of water-dispersible carriers include water-dispersible clays such as that sold under the Trade Mark ‘Laponite XLG’, starch, for example granulated starch or native starch, calcium sulphate, calcium carbonate, synthetic calcium silicate. Soft carriers are preferred to hard carriers, so that the granulated shampoo composition feels soft to the touch even before it has been contacted with water. The carrier may comprise a mixture of different carriers, for example sodium sulfate and starch or sodium acetate and starch and clay (laponite) for improved solubility in water. Because of the granulation process used, a great variety of solid particulate carriers can be chosen. Simple and cheap solid particulate carriers can be used, avoiding the need for special, expensive and complicated to produce carriers like destructurized starch.

The carrier can alternatively be water-insoluble. Examples of water-insoluble carriers which can be used in the process of the invention include zeolites, for example Zeolite 4A or Zeolite X, and other aluminosilicates or silicates, for example magnesium silicate.

The mean particle size of a water-soluble or water-dispersible carrier which contacts the shampoo composition is generally comprised between 1 micrometer and 250 micrometer. Preferably, a water-dispersible carrier has a mean particle size between 1 and 100 micrometer, for example in the range from 2 up to 10 or 20 micrometer or in the range 65 to 90 micrometer. The water-soluble or water-dispersible carrier aids in the rapid dissolution of the liquid shampoo composition, typically in less than a minute, when the granulated shampoo is applied to hair or skin and contacted with water. A water-soluble carrier may have a mean particle size on the higher end of the range preferably between 100 and 250 micrometer.

The mean particle size of a water-insoluble carrier is preferably no more than 30 micrometer, preferably no more than 20 micrometer, more preferably no more than 10 micrometer. More preferably, the mean particle size of the water-insoluble carrier is no more than 5 micrometer, for example between 1 and 5 micrometer.

The liquid shampoo composition is contacted with the carrier in a mixer in which droplets of the liquid shampoo composition become agglomerated with carrier particles. Contact can for example be in a granulating mixer, an extruder, a compactor or in a high shear or low shear mixer. Preferably the liquid shampoo composition is contacted with the carrier in a granulating mixer in which the agglomerated product is kept in particulate form. The granulating mixer is generally a high shear mixer such as an Eirich (trade mark) pan granulator, a Schugi (trade mark) mixer, a Paxeson-Kelly (trade mark) twin core blender, a Lodige ploughshare mixer, an Aeromatic (trade mark) fluidized bed granulator or a Pharma (trade mark) drum mixer. In most granulating mixers, the liquid composition is sprayed onto the carrier particles while the carrier is being agitated. The shampoo composition can alternatively be poured into the mixer instead of spraying.

The granulated product is collected from the granulating mixer and packaged. The product from a vertical continuous granulating mixer may be fed to a fluidised bed which cools and/or dries the granules and fluidises them for transport to a packing station. If the particle size distribution of granules at the outlet of the granulating mixer is larger than desired, including fines and oversize material, the fines can for example be recovered in a filter coupled with the fluidized bed cooler and/or in a classification unit and recycled with fresh particles feeding the mixer, and oversize material can be collected, crushed down and mixed with the granulated product in a fluidized bed.

If the shampoo composition and the carrier are agglomerated in an apparatus which does not maintain the agglomerated mixture as separate granules, for example an extruder or a compactor, the agglomerated mixture can be converted into granules by flaking, by comminuting an extruded strand or by spheronization after extrusion.

One preferred form of granulating mixer is a vertical continuous granulating mixer comprising blades rotating within a tubular housing and having an inlet for solid carrier particles and a spray inlet for the solubilised liquid shampoo composition to contact the solid particles above the blades. The blades are mounted on a substantially vertical shaft aligned with the housing and rotating within the housing. The blades have a predetermined clearance from the inner wall of the housing. Contact with the liquid agglomerates the particles into granules; the liquid acts as a binder by absorbing the kinetic energy of colliding particles. The blades maintain the solid particles and granules in motion and prevent agglomeration into granules which are too large. Examples of such vertical continuous granulating mixers are described in U.S. Pat. No. 4,767,217, EP-A-744,215 and WO-A-03/059,520. Vertical continuous granulating mixer technology has the advantage that the residence time in the mixing chamber is very short, for example about 1 second, giving the possibility of high throughput.

The ratio of the weight of liquid shampoo composition to the weight of carrier particles in the dry product can be varied within wide limits. Generally this ratio is at least 1:99 and may be up to 50:50 or even higher provided that the granules produced are stable and do not agglomerate further under the forces to which they are subjected while being transported. Preferably the ratio of the weight of liquid shampoo composition fed to the mixer to the weight of carrier particles fed to the mixer is in the range 15:75 to 50:50.

Accordingly, the weight ratio of shampoo composition to carrier in the granules produced after drying is preferably in the range 2:98 to 40:60, more preferably 4:96 to 25:75 or, in another embodiment it is in the range 25:75 to 35:65.

In addition to the surfactant, the shampoo composition may contain other ingredients known in shampoo formulations.

The composition preferably contains a conditioner. A hair conditioner is a hair care product that alters the texture and/or appearance of human hair to facilitate combing and/or styling of the hair and/or to improve the shine and/or softness of the hair, or add sensory feel on the skin. A conditioning agent may be useful for providing a conditioning benefit to the skin, hair and other parts of the body with keratin-containing tissue.

The granulated personal care shampoo permits to provide several benefits, including:
Moisturization/Emolliency
Skin Protection
Non-irritating/Non-drying/Mildness
Foaming and Cleaning Efficacy
Improved Deposition of skin actives ingredients
Longer-lasting Effect
Skin Feel & Aesthetics (during and after use)

Furthermore, the powder form provides convenience (easy to transport), new product format and a preservative is not mandatory.

A personal care article containing a conditioner is able to provide one or more of the following benefits:
Conditioning, including wet and dry detangling and combing, wet and dry feel, including smoothness, softness, slipperiness, Reduced flyaway/decreased static
Body, volume, fullness
Moisturization
Frizz control
Shine/luster
Reduced drying time
Colour protection/retention
Heat protection
Strengthening
Styling
Enhanced foam/lather.

The conditioning agent useful in the present invention can comprise: a water soluble conditioning agent; an oil soluble conditioning agent; a conditioning emulsion; or any combination or permutation of the three.

Non-limiting examples of useful conditioning agents include those selected from the group consisting of petrolatum, fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters, triglycerides and mixtures thereof.

More particularly, the conditioning agent may be selected from the group consisting of paraffin, mineral oil, petrolatum, stearyl alcohol, cetyl alcohol, cetearyl alcohol, behenyl alcohol, C10-30 polyesters of sucrose, stearic acid, palmitic acid, behenic acid, oleic acid, linoleic acid, myristic acid, lauric acid, ricinoleic acid, steareth-1-100, cetereath 1-100, cholesterols, cholesterol esters, glyceryl tribehenate, glyceryl dipalmitate, glyceryl monostearate, trihydroxystearin, ozokerite wax, jojoba wax, lanolin wax, ethylene glycol distearate, candelilla wax, carnauba wax, beeswax, and silicone waxes.

The conditioner can for example be an organopolysiloxane containing siloxane units (a silicone compound) independently selected from (R₃SiO_{0.5}), (R₂SiO), (RSiO_{1.5}), or (SiO₂) siloxy units, commonly referred to as M, D, T, and Q siloxy units respectively, where R is usually an organic group.

The silicone can be any organopolysiloxane having the general formula RnSiO(4−n)/2 in which n has an average value of one to three and R is an alkyl radical of 1-20 carbon atoms, preferably 1 to 6 carbon atoms, such as methyl, ethyl, propyl, isopropyl, butyl, isobutyl, hexyl, cyclohexyl, phenyl, tolyl, and xylyl, more preferably methyl, or aryl groups such as phenyl. Illustrative polysiloxanes are polydimethylsiloxane, polydiethylsiloxane, polymethylethylsiloxane, polymethylphenylsiloxane, and polydiphenylsiloxane. The organopolysiloxane can be cyclic, linear, branched, and mixtures thereof. Some examples of the silicone compositions and emulsions containing the silicone compositions that can be used as the silicone active ingredient have been described for example in U.S. Pat. No. 4,620,878, U.S. Pat. No. 5,895,794, U.S. Pat. No. 6,013,682, U.S. Pat. No. 6,316,541, U.S. Pat. No. 6,395,790, U.S. Pat. No. 6,878,773 and EP 874,017.

In one embodiment, the silicone can be a volatile methyl siloxane (VMS) which includes low molecular weight linear and cyclic volatile methyl siloxanes. Volatile methyl siloxanes conforming to the CTFA definition of cyclomethicones are considered to be within the definition of low molecular weight siloxane.

Linear VMS have the formula (CH3)3SiO{(CH3)2SiO}fSi(CH3)3. The value of f is 0-7. Cyclic VMS have the formula {(CH3)2SiO}g. The value of g is 3-6. Preferably, these volatile methyl siloxanes have a molecular weight of less than 1,000; a boiling point less than 250° C.; and a viscosity of 0.65 to 5.0 centistoke (mm2/s), generally not greater than 5.0 centistoke (mm2/s).

Representative linear volatile methyl siloxanes are hexamethyldisiloxane (MM) with a boiling point of 100° C., viscosity of 0.65 mm2/s, and formula Me₃SiOSiMe₃; octamethyltrisiloxane (MDM) with a boiling point of 152° C., viscosity of 1.04 mm²/s, and formula Me₃SiOMe₂SiOSiMe₃; decamethyltetrasiloxane (MD₂M) with a boiling point of 194° C., viscosity of 1.53 mm²/s, and formula Me₃SiO(Me₂SiO)₂SiMe₃; dodecamethylpentasiloxane (MD₃M) with a boiling point of 229° C., viscosity of 2.06 mm²/s, and formula Me₃SiO(Me₂SiO)₃SiMe₃; tetradecamethylhexasiloxane (MD₄M) with a boiling point of 245° C., viscosity of 2.63 mm²/s, and formula Me₃SiO(Me₂SiO)₄SiMe₃; and hexadecamethylheptasiloxane (MD₅M) with a boiling point of 270° C., viscosity of 3.24 mm²/s, and formula Me₃SiO(Me₂SiO)₅SiMe₃.

Representative cyclic volatile methyl siloxanes are hexamethylcyclotrisiloxane (D3), with a boiling point of 134° C., a molecular weight of 223, and formula {(Me2)SiO}3; octamethylcyclotetrasiloxane (D4) with a boiling point of 176° C., viscosity of 2.3 mm2/s, a molecular weight of 297, and formula {(Me2)SiO}4; decamethylcyclopentasiloxane (D5) with a boiling point of 210° C., viscosity of 3.87 mm2/s, a molecular weight of 371, and formula {(Me2)SiO}5; and dodecamethylcyclohexasiloxane (D6) with a boiling point of 245° C., viscosity of 6.62 mm2/s, a molecular weight of 445, and formula {(Me2)SiO}6.

The silicone oil may also be selected from any of the volatile methyl siloxanes structures listed above where some of methyl groups are replaced with a hydrocarbon group containing 2-12 carbon atoms, such as ethyl or propyl groups, for example; [(CH₃)₃SiO]₂RSiO where R is an alkyl group such as ethyl, propyl, hexyl or octyl.

Alternatively to volatile methyl siloxanes, the silicone oil may be selected from volatile ethyl siloxanes.

The silicone oil may also be selected from one of the following volatile methyl siloxanes VMS: TM₃ structures, such as [(CH₃)₃SiO]₃SiR or [(CH₃)₃SiO]₂RSiOSiR[OSi(CH₃)₃]₂, where R is alkyl group such as methyl, ethyl, propyl, butyl, pentyl, hexyl, or cyclohexyl; QM₄ structures, such as [(CH₃)₃SiO]₄Si.

The silicone can be alkylmethylsiloxane materials. These materials include liquids and waxes. The liquids can be either cyclic having a structure comprising:

(MeRSiO)ₐ(Me₂SiO)_{b}

or linear having a structure comprising:

R′Me₂SiO(MeRSiO)_{w}(Me₂SiO)ₓSiR′Me₂

wherein each R is independently a hydrocarbon of 6 to 30 carbon atoms, R′ is methyl or R, a is 1-6, b is 0-5, w is 0-5 and x is 0-5, provided a+b is 3-6 and b is not 0 if R′ is methyl. These liquids may be either volatile or non-volatile and they can have a wide range of viscosities such as from about 0.65 to about 50,000 mm²/s.
Alkylmethylsiloxane may have the structure:

R′Me₂SiO(Me₂SiO)y(MeRSiO)zSiMe₂R′ (III)

wherein y is 0-100, z is 1-100, R is an alkyl group of 6-30 carbon atoms and R′ is methyl or R.
Preferably, the alkylmethylsiloxane has the formula:

Me₃SiO(Me₂SiO)_{y}(MeRSiO)_{z}SiMe₃ (IV)

The above alkylmethylsiloxane materials are known in the art and can be produced by known methods. They may be liquid or waxy at ambient temperature (25° C.).

The silicone may also be a silicone oil in combination with other organopolysiloxanes, such as resins, gums or elastomers. Silicone elastomers have been used extensively in personal care applications for their unique silky and powdery sensory profile. Most of these elastomers can gel volatile silicones fluids as well as low polarity organic solvents such as isododecane. Representative examples of such silicone elastomers are taught in U.S. Pat. No. 5,880,210 and U.S. Pat. No. 5,760,116, both incorporated for their teaching of suitable silicone elastomer compositions that may be used in the present invention. To improve compatibilities of silicone elastomers with various personal care ingredients, alkyls, polyether, amines or other organofunctional groups have been grafted onto the silicone elastomer backbone. Representative of such organofunctional silicone elastomers are taught in U.S. Pat. No. 5,811,487 , U.S. Pat. No. 5,880,210, U.S. Pat. No. 6,200,581, U.S. Pat. No. 5,236,986, U.S. Pat. No. 6,331,604, U.S. Pat. No. 6,262,170, U.S. Pat. No. 6,531,540 and U.S. Pat. No. 6,365,670, which are incorporated by reference for teaching of organofunctional silicone elastomers suitable in the present invention.

The silicone may be a gum. Polydiorganosiloxane gums are known in the art and are available commercially. They consist of generally insoluble polydiorganosiloxanes having a viscosity in excess of 1,000,000 centistoke (mm2/s) at 25° C., alternatively greater than 5,000,000 centistoke (mm2/s) at 25° C. These silicone gums are typically sold as compositions already dispersed in a suitable solvent to facilitate their handling. Ultra-high viscosity silicones can also be included as optional ingredients. These ultra-high viscosity silicones typically have a kinematic viscosity greater than 5 million centistoke (mm2/s) at 25° C. to about 20 million centistoke (mm2/s) at 25° C. Compositions of this type in the form of suspensions are most preferred, and are described for example in U.S. Pat. No. 6,013,682.

Silicone resins may be included in the present compositions. These resin compositions are generally highly crosslinked polymeric siloxanes. Crosslinking is obtained by incorporating trifunctional and/or tetrafunctional silanes with the monofunctional silane and/or difunctional silane monomers used during manufacture. The degree of crosslinking required to obtain a suitable silicone resin will vary according to the specifics of the silane monomer units incorporated during manufacture of the silicone resin. In general, any silicone having a sufficient level of trifunctional and tetrafunctional siloxane monomer units, and hence possessing sufficient levels of crosslinking to dry down to a rigid or a hard film can be considered to be suitable for use as the silicone resin. Commercially available silicone resins suitable for applications herein are generally supplied in an unhardened form in low viscosity volatile or non-volatile silicone fluids. The silicone resins should be incorporated into compositions of the invention in their non-hardened forms rather than as hardened resinous structures.

Silicone acrylate copolymers may be included in the present compositions. Representative examples are described in EP 0963751.

Silicone carbinol fluids may be included in the present compositions. These materials are described in WO 03/101412, and can be commonly described as substituted hydrocarbyl functional siloxane fluids or resins.

Water soluble or water dispersible silicone polyether compositions may be included in the present compositions: These are also known as polyalkylene oxide silicone copolymers, silicone poly(oxyalkylene) copolymers, silicone glycol copolymers, or silicone surfactants. These can be linear rake or graft type materials, or ABA and ABn types where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible.

The silicone component may comprise a silicone material having at least one nitrogen containing substituent. Although silicone materials may be silanes, preferably the silicone material is a siloxane polymer having units of the general formula RaSiO4-a/2, wherein each R is independently selected from hydrocarbon groups having from 1 to 12 carbon atoms, preferably alkyl, alkenyl, alkynyl, aryl, alkaryl or aralkyl and a has a value of from 0 to 3, and units of the general formula RbR′SiO3-b/2, where R is as defined above, R′ is a nitrogen containing group and b has a value of from 0 to 2. Preferably R is an alkyl group having from 1 to 6 carbon atoms or an aryl or substituted aryl group having from 6 to 8 carbon atoms, such as methyl, ethyl, propyl, isopropyl, butyl, isobutyl, hexyl, cyclohexyl, phenyl, tolyl, and xylyl. Preferably the nitrogen in R′ is part of an amino functionality, amido functionality, imide functionality or quaternary ammonium functionality and most preferably amino or amido functionality. These are well known and have been described in many patent applications.

Suitable silicone materials include polyorganosiloxanes of the unit general formula RₙSiO_{4-n/2} wherein n has an average value of from 1.9 to 2.1 and R represents an organic radical attached to silicon through a silicon to carbon bond, from 0.25 to 50 per cent of the R substituents being monovalent radicals having less than 30 carbon atoms and containing, in a position at least 3 carbon atoms distance from the silicon atom, at least one —NH— radical and/or at least one —NHX radical, wherein X represents a hydrogen atom, an alkyl radical of 1 to 30 carbon atoms or an aryl radical, the remaining R substituents being monovalent hydrocarbon radicals, halogenated hydrocarbon radicals, carboxyalkyl radicals or cyanoalkyl radicals of 1 to 30 carbon atoms, at least 70 per cent of these remaining R substituents being monovalent hydrocarbon radicals of from 1 to 18 inclusive carbon atoms. In the polyorganosiloxanes at least 0.25 per cent and up to 50 per cent of the total R substituents may consist of the specified amino containing monovalent radicals. The preferred polyorganosiloxanes are, however, those in which the amino-containing substituents comprise from 1 to 5 per cent of the total R substituents.

Preferably also the alkyl and aryl radicals represented by X are those having less than 19 carbon atoms and are e.g. methyl, ethyl, propyl, butyl, nonyl, tetradecyl and octadecyl, aryl radicals e.g. phenyl and naphtyl aralkyl radicals e.g. benzyl and beta-phenylethyl, alkaryl, e.g. ethylphenyl and alkenyl e.g. vinyl and allyl. A proportion of the remaining R substituents may be other than monovalent hydrocarbon radicals, for example hydrogen atoms, halogenated hydrocarbon radicals, e.g. chlorophenyl and other substituted hydrocarbon radicals, e.g. carboxyalkyl and cyanoalkyl. However, preferably substantially all of the remaining R substituents are methyl radicals. The amino-containing substituents may contain up to 30, preferably from 3 to 11, carbon atoms. The nitrogen atom of any amino radical in R is linked to the silicon atom through a chain of at least 3 carbon atoms.

Examples of the operative amino-containing substituents are the —(CH₂)₃NH₂, —(CH₂)₃NHCH₂CH₂NH₂, —CH₂CH.CH₃.CH₂NHCH₂CH₂NH₂ and —(CH₂)₃NH(CH₂)₆NH.CH₃ radicals. Also operative are polyalkyleneimine radicals, e.g. those of the general formula R″₂NCH₂CH₂(NHCH₂CH₂)ₓNH₃R′— where R″ is a hydrogen atom, an alkyl radical or an aryl radical, x has a value from 1 to 10 inclusive, y is 1 or 2 and R′ is a saturated divalent or trivalent hydrocarbon radical having at least 3 carbon atoms. The preferred polyorganosiloxanes therefore include copolymers of dimethvlsiloxane units with delta-aminobutyl(methyl)siloxane units or gamma-aminopropyl(methyl)siloxane units, copolymers of dimethylsiloxane units with methyl(N-beta-aminoethyl-gamma-aminopropyl) siloxane units and copolymers of dimethvlsiloxane units with methyl(N-betaaminoethyl-gamma-aminoisobutyl) siloxane units. If desired the copolymers may be end-stopped with suitable chain terminating units, for example trimethylsiloxane units, dimethylphenylsiloxane units or dimethylvinylsiloxane units. Also if desired at least some of the amino-containing substituents may be present in the chain terminating units.

Suitable are also polydiorganosiloxanes which may be linear (unbranched) or substantially linear siloxane polymers having at least one silicon-bonded —Ru*X group in the molecule. The group R* is a divalent moiety, such as alkylene, alkenylene, arylene, or substituted alkylene, alkenylene or arylene, X may be NQC(O)R′ wherein Q represents hydrogen, alkyl, alkenyl, aryl or substituted alkyl, alkenyl or aryl, R′ represents e.g. H, methyl, ethyl, propy], octyl, steary], vinyl or phenyl, or may be —C(O)NR″₂ wherein R″ represents e.g. hydrogen, methyl, ethyl, butyl, octyl, dodecyl, octadecyl or phenyl, or may be the group —[NZ(CH₂)ₙ]ₚ NZ(CH₂)ₙNZQ, wherein Z represents hydrogen or R′C(O)—, n is an integer of from 2 to 6 and p is 0, 1 or 2. Examples of X groups therefore are NH.C(O)CH₃; —NHC(O)C₄H₉; —NH.C(O)C₈H₁₇; —C(O)NH₂; —C(O)NH(C₄H₉); —C(O)NH(C₁₈H₃₇); —C(O)N(C₂H₅)₂; —NC(O)CH₃(CH₂)₂NHC(O)CH₃; —NH(CH₂)₂NHC(O)CH₃; —NC(O)CH₃N(CH₂)₆NC(O)C₂H₅; —NH(CH₂)₂NHC(O)C₁₇H₃₅; —NH(CH₂)₄MC(O)C₆H═ and —NH(CH₂)₂NC(O)CH₃.(CH₂)₂NHC(O)CH₃. At least 50 percent of the silicon-bonded substituents in the polydiorganosiloxane may be methyl groups, any substituents present in addition to the —RX groups and the methyl groups being monovalent hydrocarbon groups having from 2 to 20 carbon atoms or the groups —RNH₂, —RCOOH and —R[NH(CH₂)ₙ]ₚNH(CH₂)ₙNH₂. The exemplified polydiorganosiloxane may comprise 1% RX groups of the total number of substituents in the polydiorganosiloxane. The polydiorganosiloxanes are preferably terminated with triorganosiloxy, e.g. trimethylsiloxy, groups but may be terminated with groups such as hydroxy or alkoxy. Although the polydiorganosiloxanes are preferably those consisting of diorganosiloxane units, with or without triorganosiloxane units, they may contain small proportions of chain-branching units, that is mono-organosiloxy units, and Si0₂ units. The molecular size of the suitable polydiorganosiloxanes is not critical and they may vary from freely flowing liquids to gummy solids. The preferred polydiorganosiloxanes are, however, those having a viscosity in the range from about 5.10⁻⁵ to about 5.10⁻² m²/s at 20° C. Such polydiorganosiloxanes are more easily emulsified than the higher viscosity materials. Suitable preparative methods are known in the art and are described for example in GB 882 059, GB 882 061, GB 788 984 and GB 1 117 043.

Suitable aminosilanes have the general formula R′_{z}Si(OR)_{4-z} where R can be an alkyl group such as methyl, ethyl, n-propyl, isopropyl, and t-butyl or an aromatic group such as phenyl, tolyl, and xylyl, but is preferably methyl. R′ is an amine-containing group, and z is an integer with a value of 1 to 3, preferably 1 or 2. R′ has the general formula —R⁸R⁷, wherein each R⁷ is independently selected from the group consisting of a hydrogen atom and a group of the formula —R⁸NH₂, and each R⁸ is independently a divalent hydrocarbon group. Typically, R′ is an aminoalkyl group, such as —(CH₂)_{w}NH₂ or —(CH₂)_{w}NH—(CH₂)_{w}NH₂, wherein w is an integer, preferably with a value of 2 to 4. Examples of suitable aminosilanes include aminoethylaminoisobutylmethyldimethoxysilane, (ethylenediaminepropyl)-trimethoxysilane, and gammaaminopropyltriethoxysilane. Aminosilanes are known in the art and are commercially available. U.S. Pat. No. 5,117,024 discloses aminosilanes and methods for their preparation.

The conditioning agent may be an organosilicon component of the formula Si(OZ)₄, ZSi(OZ′)₃ or Z₂Si(OZ′)₂ in which Z represents an alkyl, substituted alkyl, aryl or substituted aryl group having 1 to 20 carbon atoms and each Z′ represents an alkyl group having 1 to 6 carbon atoms. Preferably Z represents an alkyl, substituted alkyl, aryl or substituted aryl group having 6 to 18 carbon atoms.

The organosilicon component may comprise a condensation compound obtained by the hydrolysis-condensation of any combination of compounds of the formula Si(OZ)₄, ZSi(OZ')₃ or Z₂Si(OZ)₂, in which Z represents an alkyl, substituted alkyl, aryl or substituted aryl group having 1 to 20 carbon atoms and each Z′ represents an alkyl group having 1 to 6 carbon atoms.

Preferably, the organosilicon component comprises alkoxysilyl groups having 1 or 2 carbon atoms, preferably 1 carbon atom (methoxysilyl groups).

The organosilicon component can contain an organopolysiloxane. This may be chosen from any known organopolysiloxane materials, i.e. materials which are based on a Si—O—Si polymer chain and which may comprise mono-functional, di-functional, tri-functional and/or tetra-functional siloxane units, many of which are commercially available. It is preferred that the majority of siloxane units are di-functional materials having the general formula RR′SiO_{2/2}, wherein R or R′ independently denotes an organic component or an amine, hydroxyl, hydrogen or halogen substituent. Preferably R will be selected from hydroxyl groups, alkyl groups, alkenyl groups, aryl groups, alkyl-aryl groups, aryl-alkyl groups, alkoxy groups, aryloxy groups and hydrogen. More preferably a substantial part, most preferably a majority of the R substituents will be alkyl groups having from 1 to 12 carbon atoms, most preferably methyl or ethyl groups. The organopolysiloxane can for example be polydimethylsiloxane (PDMS). Alternatively the organopolysiloxane may comprise methylalkylsiloxane units in which the said alkyl group contains 2-20 carbon atoms. Such methylalkylsiloxane polymers, particularly those in which the said alkyl group contains 6-20 carbon atoms, may confer even higher water resistance than PDMS. Blends of organopolysiloxanes can be used, for example a blend of a methylalkylsiloxane polymer with a linear PDMS.

In a preferred embodiment, the organosilicon component comprises a dialkoxysilane, trialkoxysilane, or a mixture of these with each other or with an organopolysiloxane. The dialkoxysilane generally has the formula Z₂Si(OZ′)₂ and the trialkoxysilane generally has the formula ZSi(OZ′)₃ in which Z in each formula represents an alkyl, substituted alkyl, aryl or substituted aryl group having 1 to 20 carbon atoms and each Z′ represents an alkyl group having 1 to 6 carbon atoms. The group Z can for example be substituted by a halogen, particularly fluoro, group, an amino group or an epoxy group, or an alkyl group can be substituted by a phenyl group or a phenyl group can be substituted by an alkyl group. Preferred silanes include those in which Z represents an alkyl group having 6 to 18 carbon atoms and each Z′ represents an alkyl group having 1 to 4, particularly 1 or 2, carbon atoms, for example n-octyl trimethoxysilane, 2-ethylhexyl triethoxysilane or n-octyl trimethoxysilane.

Suitable silicone quaternary ammonium compounds are disclosed by U.S. Pat. No. 5,026,489 entitled, “Softening Compositions Including Alkanolamino Functional Siloxanes.” The patent discloses monoquaternary ammonium functional derivatives of alkanolamino polydimethylsiloxanes. The derivatives are exemplified by (R⁹₃SiO)₂SiR⁹—(CHR¹⁰)ₐNR¹⁰_{b}R¹¹_{3-b} wherein R⁹ is an alkyl group, R¹⁰ is H, alkyl, or aryl, R¹¹ is (CHR¹⁰)OH, a is 1 to 10, and b is 1 to 3.

The silicone can be a saccharide-siloxane copolymer having a saccharide component and an organosiloxane component and linked by a linking group. The saccharide-siloxane copolymer has the following formula:

R²ₐR¹₍₃₋ₐ₎SiO—[(SiR²R¹O)ₘ—(SiR¹₂O)ₙ]_{y}—SiR¹₍₃₋ₐ₎R²ₐ

wherein each R¹ can be the same or different and comprises hydrogen, C₁-C₁₂ alkyl, an organic radical, or R³-Q, Q comprises an epoxy, cycloepoxy, primary or secondary amino, ethylenediamine, carboxy, halogen, vinyl, allyl, anhydride, or mercapto functionality, m and n are integers from 0 to 10,000 and may be the same or different, each a is independently 0, 1, 2, or 3, y is an integer such that the copolymer has a molecular weight less than 1 million, R² has the formula Z-(G¹)_{b}-(G²)_{c}, and there is at least one R² per copolymer, wherein G¹ is a saccharide component comprising 5 to 12 carbons, b+c is 1-10, b or c can be 0, G² is a saccharide component comprising 5 to 12 carbons additionally substituted with organic or organosilicon radicals, Z is the linking group and is independently selected from the group consisting of:
R³—NHC(O)—R⁴—;
R³—NHC(O)O—R⁴—;
R³—NH—C(O)—NH—R⁴—;
R³—C(O)—O—R⁴—;
R³—O—R⁴—;
R³—CH(OH)—CH₂—O—R⁴—;
R³—S—R⁴
R³—CH(OH)—CH₂—NH—R⁴—; and
R³—N(R¹)—R⁴, and

R³ and R⁴ are divalent spacer groups comprising (R⁵)ᵣ(R⁶)ₛ(R⁷)ₜ, where at least one of r, s and t must be 1, and R⁵ and R⁷ are either C₁-C₁₂ alkyl or ((C₁-C₁₂)O)ₚ where p is any integer 1-50 and each (C₁-C₁₂)O may be the same or different, R⁶ is —N(R⁸)—, where R⁸ is H or C₁-C₁₂ alkyl, or is Z—X where Z is previously defined or R3.

X is a carboxylic acid, phosphate, sulfate, sulfonate or quaternary ammonium radical, and at least one of R³ and R⁴ must be present in the linking group and may be the same or different,

and
wherein the saccharide-siloxane copolymer is a reaction product of a functionalized organosiloxane polymer and at least one hydroxy-functional saccharide such that the organosiloxane component is covalently linked via the linking group, Z, to the saccharide component.

The organopolysiloxane may contain any number or combination of M, D, T, or Q units, but has at least one substituent that is a sulfonate group having the general formula:

R¹-G-(CO)-Ph-SO3⁻M⁺

where;

R¹ is a divalent organic group bonded to the organopolysiloxane; M is hydrogen, an alkali metal, or a quaternary ammonium; G is an oxygen atom, NH, or an NR group where R is a monovalent organic group, and. Ph is a phenyl cycle.

The sulfonate group substituent is bonded to the organopolysiloxane via a Si—C bond by the R¹ moiety. The sulfonate group substituent can be present in the organopolysiloxane via linkage to any organosiloxy unit, that is, it may be present on any M, D, or T siloxy unit. The sulfonate functional organopolysiloxane can also contain any number of additional M, D, T, or Q siloxy units of the general formula (R₃SiO_{0.5}), (R₂SiO), (RSiO_{1.5}), or (SiO₂), where R is a monovalent organic group, providing that the organopolysiloxane has at least one siloxy unit with the sulfonate functional group present.

The monovalent organic groups represented by R in the organopolysiloxanes may have from 1 to 20 carbon atoms, alternatively 1 to 10 carbon atoms, and are exemplified by, but not limited to alkyl groups such as methyl, ethyl, propyl, butyl, pentyl, hexyl, heptyl, octyl, undecyl, and octadecyl; cycloalkyl such as cyclohexyl; aryl such as phenyl, tolyl, xylyl, benzyl, and 2-phenylethyl; amine functional organic groups such as aminopropyl and aminoethylaminoisobutyl; a polyalkylene oxide (polyether) such as polyoxyethylene, polyoxypropylene, polyoxybutylene, or mixtures thereof, and halogenated hydrocarbon groups such as 3,3,3-trifluoropropyl, 3-chloropropyl, and dichlorophenyl. Typically, at least 50 percent, alternatively at least 80%, of the organic groups in the organopolysiloxane may be methyl (denoted as Me).

The R¹ group in the sulfonate group substituent can be any divalent organic group, but typically is a divalent hydrocarbon group containing 2 to 6 carbon atoms. Divalent hydrocarbons are represented by an ethylene, propylene, butylene, pentylene, or hexylene group. Alternatively, R¹ is a propylene group, —CH.₂CH.₂CH.₂— or an isobutylene group, —CH₂CH(CH₃)CH₂—.

G in the general formula for the sulfonate substituent group above is an oxygen atom, NH, or an NR group where R is a monovalent organic group. When G is an NR group, R can be any of the monovalent organic groups described above. Typically, G is the NH chemical unit forming an amide group in the sulfonate substituent formula above.

The conditioner is preferably mixed with the surfactant in the liquid shampoo composition before the shampoo is formed into granules. An organopolysiloxane conditioner, for example, can be in the form of a pure fluid or an emulsion or a suspension when it is mixed into the shampoo composition. Where an emulsion or suspension is used, the water present in the emulsion or suspension forms some or all of the water required to solubilise the surfactants present in the shampoo composition. Suitable polydiorganosiloxane emulsions are described for example in EP-A-432951, EP-A-798332, U.S. Pat. No. 6,013,682, EP-A-1263840 and EP-A-1054032.

The shampoo composition can contain other ingredients selected for example from perfumes, fragrances, colorants such as dyes, essential oils, vitamins, deposition agents such as polyquaternary compounds to improve the deposition of active ingredients from the shampoo onto hair or skin, buffering agents, stabilizers, proteins, preservatives, anti-dandruff agent, disinfectants and antimicrobial agents. Such ingredients can be mixed into the liquid shampoo composition before granulation or they can be mixed to the granulated shampoo.

### Other Additives

Other additives can include, depending on the use, glycols, vitamins A and E in their various forms, sunscreen agents, humectants, oil components, styling agents, preservatives, such as known parabens, emollients, occlusive agents, and esters. Other optional components may be added to the shampoo compositions of this invention such as fragrances, preservatives, vitamins, ceramides, amino-acid derivatives, antioxidants, electrolytes, liposomes, polyols, such as glycerine and propylene glycol and botanicals (plant extracts)”

### Anti-Dandruff Agents

These agents include particulate antidandruff agents such as pyridinethione salts, selenium compounds such as selenium disulfide, and soluble antidandruff agents.

### Colorants/Coloration

Oxidation hair dyeing agents are most widely used as permanent hair dyeing agents. Oxidation dye precursors in such hair dyeing agents penetrate into hair, and chemically impart a colour to the hair by means of colour formation resulting from oxidative polymerisation under the action of an oxidation agent. Non-oxidation dyeing agents are used for semi-permanent or non-permanent hair dyeing. Semi-permanent or non-oxidation dyeing agents are sometimes also referred to as direct dyes. Semi-permanent dyeing will usually colour human hair for up to six subsequent shampoo washes, although a high proportion of colour is often lost after 2 or 3 washes. Semi-permanent hair dyeing compositions are usually provided as single-component products, and may contain a variety of additives in addition to a direct dye. Preferably, the personal care product containing an oxidation dye precursor is in the form of a powder-like single-component product.

### Conditioning Agent (Additional)

Additional conditioners, other than the silicone component, may be added to the shampoo composition in the form of organic cationic conditioning agents for the purpose of providing more hair grooming. Such cationic conditioning agents may include quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats Polyquaternium-7, Polyquaternium-8, Polyquaternium-10, Polyquaternium-11, and Polyquaternium-23. The above cationic organic polymers and others are described in more details in U.S. Pat. No. 4,240,450 which is hereby incorporated by reference to further describe the cationic organic polymers. Other categories of conditioners such as cationic surfactants such as cetyl trimethylammonium chloride, cetyl trimethylammonium bromide, and stearyltrimethylammonium chloride, may also be employed in the compositions as a cationic conditioning agent.

### Deposition Agents

Cationic deposition aid, preferably a cationic deposition polymer can be present in the composition. The polymer may be a homopolymer or be formed from two or more types of monomers. The molecular weight of the polymer will generally be between 5 000 and 10 000 000, typically at least 10 000 and preferably in the range 100 000 to about 2 000 000. The polymers will have cationic nitrogen containing groups such as quaternary ammonium or protonated amino groups, or a mixture thereof. The cationic charge density has been found to need to be at least 0.1 meq/g, preferably above 0.8 or higher. The cationic charge density should not exceed 4 meq/g, it is preferably less than 3 and more preferably less than 2 meq/g. The charge density can be measured using the Kjeldahl method and should be within the above limits at the desired pH of use, which will in general be from about 3 to 9 and preferably between 4 and 8. The cationic nitrogen-containing group will generally be present as a substituent on a fraction of the total monomer units of the cationic deposition polymer. Thus when the polymer is not a homopolymer it can contain spacer noncationic monomer units. Such polymers are described in the CTFA Cosmetic Ingredient Directory, 3rd edition. Suitable cationic deposition aids include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol. The cationic amines can be primary, secondary or tertiary amines, depending upon the particular species and the pH of the composition. In general secondary and tertiary amines, especially tertiary, a-re preferred. Amine substituted vinyl monomers and amines can be polymerized in the amine form and then converted to ammonium by quaternization. Suitable cationic amino and quaternary ammonium monomers include, for example, vinyl compounds substituted with dialkyl aminoalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternised pyrrolidine, e.g., alkyl vinyl imidazolium, and quaternised pyrrolidine, e.g., alkyl vinyl imidazolium, alkyl vinyl pyridinium, alkyl vinyl pyrrolidine salts. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide. The cationic deposition aids can comprise mixtures of monomer units derived from amine- and/or quaternary ammonium-substituted monomer and/or compatible spacer monomers. Suitable cationic deposition aids include, for example: copolymers of 1-vinyl-2-pyrrolidine and 1-vinyl-3-methylimidazolium salt (e.g., Chloride salt) (referred to in the industry by the Cosmetic, Toiletry, and Fragrance Association, “CTFA”. as Polyquaternium-16) such as those commercially available from BASF Wyandotte Corp. (Parsippany, N.J., USA) under the LUVIQUAT tradename (e.g., LUVIQUAT FC 370); copolymers of 1-vinyl-2-pyrrolidine and dimethylaminoethyl methacrylate (referred to in the industry by CTFA as Polyquaternium-11) such as those commercially from Gar Corporation (Wayne, N.J., USA) under the GAFQUAT tradename (e.g., GAFQUAT 755N); cationic diallyl quaternary ammonium-containing polymer including, for example, dimethyldiallyammonium chloride homopolymer and copolymers of acrylamide and dimethydiallyammonium chloride, referred to in the industry (CTFA) as Polyquaternium 6 and Polyquaternium 7, respectively; mineral acid salts of aminoalkyl esters of homo-and co-polymers of unsaturated carboxylic acids having from 3 to 5 carbon atoms, as described in U.S. Pat. No. 4,009,256; and cationic polyacrylamides as described in our co-pending UK Application No. 9403156.4 (W095/22311). Other cationic deposition aids that can be used include polysaccharide polymers, such as cationic cellulose derivatives and cationic starch derivatives. Cationic polysaccharide polymer materials suitable for use in compositions of the invention include those of the formula:

A-O(R—N⁺R¹R²R³X⁻)

wherein: A is an anhydroglucose residual group, such as starch or cellulose anhydroglucose residual, R is an alkylene oxyalkylene, polyoxyalkylene, or hydroxyalkylene group, or combination thereof, R1, R2 and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less, and X is an anionic counterion, as previously described. Cationic cellulose is available from Amerchol Corp. (Edison, N.J., USA) in their Polymer iR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200. Other cationic deposition aids that can be used include cationic guar gum derivatives, such as guar hydroxypropyltrimonium chloride (Commercially available from Celanese Corp. in their Jaguar trademark series). Other materials include quaternary nitrogen-containing cellulose ethers (e.g., as described in U.S. Pat. No. 3,962,418, incorporated by reference herein), and copolymers of etherified cellulose and starch (e.g., as described in U.S. Pat. No. 3,958,581, incorporated by reference herein). The deposition agent can be put in the liquid shampoo composition or added in solid form as co-carrier.

### Foam Booster

A foam booster is an agent which increases the amount of foam available from a system at a constant molar concentration of surfactant, in contrast to a foam stabilizer which delays the collapse of a foam. Foam building is provided by adding to the aqueous media, a foam boosting effective amount of a foam booster. The foam boosting agent is preferably selected from the group consisting of fatty acid alkanolamides and amine oxides. The fatty acid alkanolamides are exemplified by isostearic acid diethanolamide, lauric acid diethanolamide, capric acid diethanolamide, coconut fatty acid diethanolamide, linoleic acid diethanolamide, myristic acid diethanolamide, oleic acid diethanolamide, stearic acid diethanolamide, coconut fatty acid monoethanolamide, oleic acid monoisopropanolamide, and lauric acid monoisopropanolamide. The amine oxides are exemplified by N-cocodimethylamine oxide, N-lauryl dimethylamine oxide, N-myristyl dimethylamine oxide, N-stearyl dimethylamine oxide, N-cocamidopropyl dimethylamine oxide, N-tallowamidopropyl dimethylamine oxide, bis(2-hydroxyethyl) C12-15 alkoxypropylamine oxide. Preferably a foam booster is selected from the group consisting of lauric acid diethanolamide, N-lauryl dimethylamine oxide, coconut acid diethanolamide, myristic acid diethanolamide, and oleic acid diethanolamide. Other foam boosting agents are saponine and lecithine. The foam boosting agent is preferably present in the shampoo compositions of this invention in an amount from about 0.5 to 15 wt % and more preferably about 1 to 10 wt % based on the total weight of the dry composition.

The composition may further comprise a polyalkylene glycol to improve lather performance. Concentration of the polyalkylene glycol in the shampoo composition may range from about 0.01% to about 15%, preferably from about 0.05% to about 10%, and more preferably from about 0.1% to about 8%, by weight of the dry composition. The optional polyalkylene glycols are characterized by the general formula:

H(OCH2CHR)n-OH

wherein R is selected from the group consisting of H, methyl, and mixtures thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist. In the above structure, the molecular weight has an average value of from about 200 to about 25,000, preferably from about 2500 to about 20,000, and more preferably from about 3500 to about 15,000. Other useful polymers include the polypropylene glycols and mixed polyethylene/polypropylene glycols.

### Proteins

Hair care shampoos can contain proteins, like those extracted from wheat, soy, rice, corn, keratin, elastin or silk. Most are in the hydrolyzed form and they can also be quaternised to provide better performance.

### Fragrances

Another type of active ingredient that can be included in the composition is a perfume or fragrance. The perfume can be a fragrant odoriferous substance or a mixture of fragrant odoriferous substances including natural substances obtained by extraction of flowers, herbs, leaves, roots, barks, wood, blossoms or plants; artificial substances including mixtures of different natural oils or oil constituents; and synthetically produced substances. Some examples of perfume ingredients that are useful include hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; and undecalactone gamma.

Additional examples of perfume ingredients include orange oil; lemon oil; grapefruit oil; bergamot oil; clove oil; dodecalactone gamma; methyl-2-(2-pentyl-3-oxo-cyclopentyl) acetate; beta-naphthol methylether; methyl-beta-naphthylketone; coumarin; decylaldehyde; benzaldehyde; 4-tert-butylcyclohexyl acetate; alpha, alpha-dimethylphenethyl acetate; methylphenylcarbinyl acetate; Schiff's base of 4-(4-hydroxy-4-methylpentyl)-3-cyclohexene-1-carboxaldehyde and methyl anthranilate; cyclic ethyleneglycol diester of tridecandioic acid; 3,7-dimethyl-2,6-octadiene-1-nitrile; ionone gamma methyl; ionone alpha; ionone beta; petitgrain; methyl cedrylone; 7-acetyl-1,2,3,4,5,6,7,8-octahydro-1,1,6,7-tetramethyl-naphthalene; ionone methyl; methyl-1,6,10-trimethyl-2,5,9-cyclododecatrien-1-yl ketone; 7-acetyl-1,1,3,4,4,6-hexamethyl tetralin; 4-acetyl-6-tert-butyl-1,1-dimethyl indane; benzophenone; 6-acetyl-1,1,2,3,3,5-hexamethyl indane; 5-acetyl-3-isopropyl-1,1,2,6-tetramethyl indane; 1-dodecanal; 7-hydroxy-3,7-dimethyl octanal; 10-undecen-1-al; iso-hexenyl cyclohexyl carboxaldehyde; formyl tricyclodecan; cyclopentadecanolide; 16-hydroxy-9-hexadecenoic acid lactone; 1,3,4,6,7,8-hexahydro-4,6,6,7,8,8-hexamethylcyclopenta-gamma-2-benzopyrane; ambroxane; dodecahydro-3a,6,6,9a-tetramethylnaphtho-2,1b furan; cedrol; 5-(2,2,3-trimethylcyclopent-3-enyl)-3-methylpentan-2-ol; 2-ethyl-4-(2,2,3-trimethyl-3-cyclopenten-1-yl)-2-buten-1-ol; caryophyllene alcohol; cedryl acetate; para-tert-butylcyclohexyl acetate; patchouli; olibanum resinoid; labdanum; vetivert; copaiba balsam; fir balsam; and condensation products of: hydroxycitronellal and methyl anthranilate; hydroxycitronellal and indol; phenyl acetaldehyde and indol; 4-(4-hydroxy-4-methyl pentyl)-3-cyclohexene-1-carboxaldehyde, and methyl anthranilate.

More examples of perfume ingredients are geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methylether; isolongifolanone; aubepine nitrile; aubepine; heliotropine; eugenol; vanillin; diphenyl oxide; hydroxycitronellal ionones; methyl ionones; isomethyl ionomes; irones; cis-3-hexenol and esters thereof; indane musk fragrances; tetralin musk fragrances; isochroman musk fragrances; macrocyclic ketones; macrolactone musk fragrances; and ethylene brassylate.

### Pediculicides

Pediculicides, for control of lice infestations. Suitable pediculicides are well known in the art and include, for example, pyrethrins such as those described in U.S. Pat. No. 4,668,666, which description is incorporated herein by reference in its entirety.

### PH Control Agents

A pH adjusting agent, preferably to adjust the pH within the range of 4 to 9 and more preferably within the range of 5 to 7. Any water soluble acid such as a carboxylic acid or a mineral acid is suitable. For example, suitable acids include mineral acids such as hydrochloric acid, sulphuric acid, and phosphoric acid, monocarboxylic acid such as acetic acid and lactic acid, and polycarboxylic acids such as succinic acid, adipic acid, and citric acid.

### Pigments and Dyes

Typical pigments are iron oxides and titanium dioxide which can be present in the composition in the amount of 0.1 to 30 wt.-%, preferably 0.5 to 20 wt.-% and most preferably 0.8 to 10 wt.-%.

### Preservatives

It may be desirable to add various preservatives such as the parabens, BHT, BHA, etc or any usual preservative. Generally, 0.01-5% preservative is suggested.

### Sunscreen

These include those which absorb ultraviolet light between about 290-320 nanometers (the UV-B region) such as, but not exclusively, para-aminobenzoic acid derivatives and cinnamates such as octyl methoxycinnamate and those which absorb ultraviolet light in the range of 320-400 nanometers (the UV-A region) such is benzophenones and butyl methoxy dibenzoylmethane. Some additional examples of sunscreen chemicals which may be employed in accordance with the present invention are 2-ethoxyethyl p-methoxycinnamate; menthyl anthranilate; homomenthyl salicylate; glyceryl p-aminobenzoate; isobutyl p-aminobenzoate; isoamyl p-dimethylaminobenzoate; 2-hydroxy-4-methoxybenzophenones sulfonic acid; 2,2′-dihydroxy-4-methoxybenzophenone; 2-hydroxy-4-methoxybenzophenone; 4-mono and 4-bis(3-hydroxy-propyl)amino isomers of ethyl benzoate; and 2-ethylhexyl p-dimethylaminobenzoate

### Vitamins

Vitamins are a class of organic compounds that must be ingested part of the diet for humans (and other organisms) in order to maintain health and well being. Some vitamins also have beneficial effects when applied topically and for this reason are popular ingredients in various personal care formulations, where it is desired that the vitamin should be released after the formulation has been applied to the skin or hair.

Vitamins comprise a variety of different organic compounds such as alcohols, acids, sterols, and quinones. They can be classified into two solubility groups: lipid-soluble vitamins and water-soluble vitamins. Lipid-soluble vitamins that have utility in personal care formulations include retinol (vitamin A), ergocalciferol (vitamin D₂), cholecalciferol (vitamin D₃), phytonadione (vitamin K₁), and tocopherol (vitamin E). Water-soluble vitamins that have utility in personal care formulations include ascorbic acid (vitamin C), thiamin (vitamin B₁) niacin (nicotinic acid), niacinamide (vitamin B₃), riboflavin (vitamin B₂), pantothenic acid (vitamin B₅), biotin, folic acid, pyridoxine (vitamin B₆), and cyanocobalamin (vitamin B₁₂).

Many of the vitamins that are used in personal care compositions are inherently unstable and therefore present difficulties in the preparation of shelf-stable personal care compositions. The instability of the vitamins is usually related to their susceptibility to oxidation. For this reason, vitamins are often converted into various derivatives that are more stable in personal care formulations. These vitamin derivatives offer other advantages in addition to improved stability. Vitamin derivatives can be more amenable to certain kinds of personal care formulations. For example a lipid-soluble vitamin can be derivatised to produce a water-soluble material that is easier to incorporate into a water-based formulation. Retinol and tocopherol are two lipid-soluble vitamins that are particularly useful in skin care compositions and consequently there are many different derivatives of these two vitamins that are used in personal care compositions. Derivatives of retinol include retinyl palmitate (vitamin A palmitate), retinyl acetate (vitamin A acetate), retinyl linoleate (vitamin A linoleate), and retinyl propionate (vitamin A propionate). Derivatives of tocopherol include tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), and sodium tocopheryl phosphate. Derivatives of ascorbic acid (Vitamin C) such as ascorbyl palmitate, ascorbyl dipalmitate, ascorbyl glucoside, ascorbyl tetraisopalmitate, and tetrahexadecyl ascorbate can also be used as the active material, as can vitamin derivatives incorporating two different vitamins in the same compound, for example ascorbyl tocopheryl maleate, potassium ascorbyl tocopheryl phosphate or tocopheryl nicotinate.

### Foam Control Agents

Foam control agents/antifoams may be used as additives. They generally comprise a polyorganosiloxane fluid and preferably also a hydrophobic particulate filler. The polysiloxane fluid may be a substantially linear polydiorganosiloxane or may be branched as described for example in EP-A-217501, U.S. Pat. No. 5,674,938 and U.S. Pat. No. 6,150,488. The organic groups in the polyorganosiloxane fluid generally comprise methyl groups and may additionally comprise a silicon-bonded substituent of the formula Y-Ph, wherein Y denotes a divalent aliphatic organic group bonded to silicon through a carbon atom and Ph denotes an aromatic group, examples of such fluids being described in EP-A-1075864, or a higher (C8+) alkyl group, examples of such fluids being described in EP-A-578423. A preferred hydrophobic filler is silica, made hydrophobic by treatment with a methyl substituted organo-silicon material such as polydimethylsiloxane, hexamethyldisilazane, hexamethyldisiloxane or an organosilicon resin comprising monovalent groups (CH₃)₃SiO_{1/2,} or with a fatty acid, preferably at a temperature of at least 80° C. Alternative hydrophobic fillers include titania, ground quartz, alumina, aluminosilicates, organic waxes, e.g. polyethylene wax or microcrystalline wax, and/or alkyl amides such as ethylenebisstearamide or methylenebisstearamide. The silicone antifoam preferably also contains a silicone resin, for example a MQ resin comprising groups of the formula R*₃SiO_{1/2} and SiO_{4/2} groups, wherein R* denotes a monovalent hydrocarbon group. The silicone resin can be soluble, partially soluble or insoluble in the polysiloxane fluid.

### Emollients

The liquid shampoo composition may optionally contain one or more water-soluble emollients including, but not limited to, lower molecular weight aliphatic diols such as propylene glycol and butylene glycol; polyols such as glycerine and sorbitol; and polyoxyethylene polymers such as polyethylene glycol 200. The specific type and amount of water soluble emollient(s) employed will vary depending on the desired aesthetic characteristics of the composition, and is readily determined by one skilled in the art.

In order to ensure adequate coverage of the carrier with most ingredients of the shampoo composition, it is preferred to treat the carrier in conditions minimizing the risk of volatilization of the components. This can be done by choosing ingredients of low volatility such as non volatile silicones, or by working at low temperature.

The granulated product has the advantage that it is stable and does not require plastic packaging to protect it from the environment, even in hot humid climates. It can be packaged in biodegradable or recyclable packs, for example in polyvinyl alcohol film sheets, polylactic acid bags, starch or in paper, for example the types of paper used for packaging soap, sugar or flour, and remains free flowing and effective as a shampoo. This allows it to be sold in single dose packages with minimized detriment to the environment.

The invention will now be described with reference to the following Examples, in which parts and percentages are by weight, unless otherwise indicated.

Mean particle size of some of the carriers is:

| | |
|---|---|
| Zeolite | 4 micrometer |
| Starch | 12.7 micrometer |
| Laponite RD & Laponite XLG | 77 micrometer |
| Dextrose M | 196 micrometer |
| Sodium sulfate | 153 micrometer |
| Synthetic calcium silicate | 18 micrometer |

### EXAMPLE 1

160 g of Empicol ESB 3 (27% active sodium laureth sulfate) (SLES) were blended with 24 g of Amonyl 380 BA (30% active cocamidopropyl betaine) (CAPB) and 12 g Comperlan KD (cocamide DEA) (CDEA). 8 g of Dow Corning 1785 (trade mark) polydimethylsiloxane emulsion were added to the surfactants solution. 62.5 g of this solution was then poured very slowly into a high shear mixer in which 100 g native starch was placed. The mixture was stirred continuously till a particulate material was obtained. The particulate material was then passed over an Aeromatic® spray granulator for 10 minutes and 50° C. The dry composition was 10.98% SLES, 1.83% CAPB, 3.05% CDEA, 1.22% silicone and 82.93% starch.

### EXAMPLE 2

160 g of Empicol ESB 3 (27% active sodium laureth sulfate) were blended with 24 g of Amonyl 380 BA (30% active cocamidopropyl betaine) and 12 g Comperlan KD (cocamide DEA). 8 g of Dow Corning 1785 (trade mark) polydimethylsiloxane emulsion were added to the surfactants solution. 83 g of this solution was then poured on 150 g zeolite 4A. The powder was then dried at 50° C. for 20 minutes. The dry composition was 9.91% SLES, 1.65% CAPB, 2.75% CDEA, 1.1% silicone and 84.58% zeolite.

### EXAMPLE 3

160 g of Empicol ESB 3 (27% active sodium laureth sulfate) were blended with 24 g of Amonyl 380 BA (30% active cocamidopropyl betaine) and 12 g Comperlan KD (cocamide DEA). 8 g of Dow Corning 1785 (trade mark) polydimethylsiloxane emulsion were added to the surfactants solution. 25 g of this solution was then poured on 100 g of dextrose monohydrate (Roquette). The powder was then dried at 55° C. for 15 minutes. The dry composition was 4.89% SLES, 0.82% CAPB, 1.36% CDEA, 0.54% silicone and 92.39% Dextrose.

### EXAMPLE 4

160 g of Empicol ESB 3 (27% active sodium laureth sulfate) were blended with 24 g of Amonyl 380 BA (30% active cocamidopropyl betaine) and 12 g Comperlan KD (cocamide DEA). 8 g of Dow Corning 1785 (trade mark) polydimethylsiloxane emulsion were added to the surfactants solution. 15 g of this solution was then poured on 100 g sodium sulfate. The powder was then dried at 55° C. for 20 minutes. The powder was then dried at 55° C. for 15 minutes. The dry composition was 3.03% SLES, 0.50% CAPB, 0.84% CDEA, 0.34% silicone and 95.29% sodium sulfate.

In each of Examples 1 to 4, granules of mean particle diameter in the range 20 to 1000 μm were produced. The softness to touch of the granules of Example 1 was appreciated as particularly attractive for a shampoo product. The pH generated when the granules were dispersed in water is given in Table 1.

TABLE 1

| Components | Example 1 | Example 2 | Example 3 | Example 4 |
|---|---|---|---|---|
| SLES | 10.98 | 9.91 | 4.89 | 3.03 |
| CAPB | 1.83 | 1.65 | 0.82 | 0.50 |
| Cocoamide DEA | 3.05 | 2.75 | 1.36 | 0.84 |
| silicone | 1.22 | 1.10 | 0.54 | 0.34 |
| Carrier | Starch | Zeolite | Dextrose | Sodium sulphate |
| | (82.93) | (84.58) | (92.39) | (95.29) |
| pH | 7.5 | 9.5 | 7 | 7.5 |

The granules were rubbed with wet hands to test their feel as shampoo. A shampoo foam was formed in Examples 1 to 4. The granules of Examples 1, 3 and 4 all provided a pleasant feel on the skin when wetted. The hardness of the granules of Example 2, based on water-insoluble zeolite, was detected.

### EXAMPLE 5

160 g of Empicol ESB 3 (27% active sodium laureth sulfate) were blended with 24 g of Amonyl 380 BA (30% active cocamidopropyl betaine) and 12 g Comperlan KD (cocamide DEA). 50.3 g of this solution was then poured on 80 g native starch. The powder was then dried at 50° C. for 20 minutes. The dry composition was 11.55% SLES, 1.92% CAPB, 3.21% CDEA and 83.32% starch.

### EXAMPLE 6

160 g of Empicol ESB 3 (27% active sodium laureth sulfate) were blended with 24 g of Amonyl 380 BA (30% active cocamidopropyl betaine) and 12 g Comperlan KD (cocamide DEA). 22.8 g of this solution was then poured on a blend of 40 g native starch and 40 g sodium sulfate. The powder was then dried at 50° C. for 20 minutes. The dry composition was 5.76% SLES, 0.96% CAPB, 1.6% CDEA, 45.84% starch and 45.84% sodium sulfate.

### EXAMPLE 7

160 g of Empicol ESB 3 (27% active sodium laureth sulfate) were blended with 24 g of Amonyl 380 BA (30% active cocamidopropyl betaine) and 12 g Comperlan KD (cocamide DEA). 100 g of the surfactant were mixed with 4.5 g Sokalan PA 25 (polyacrylic acid binder). 30.6 g of this solution was then poured on a blend of 40 g native starch and 40 g sodium sulfate. The powder was then dried at 50° C. for 20 minutes. The dry composition was 7.17% SLES, 1.2% CAPB, 1.99% CDEA, 0.73% Sokalan PA25, 44.45% starch and 44.45% sodium sulfate.

### EXAMPLE 8

160 g of Empicol ESB 3 (27% active sodium laureth sulfate) were blended with 24 g of Amonyl 380 BA (30% active cocamidopropyl betaine) and 12 g Comperlan KD (cocamide DEA). 11.5 g of this solution was then poured on a blend of 80 g Glucidex IT-19. The powder was then dried at 50° C. for 20 minutes. The dry composition was 3.03% SLES, 0.5% CAPB, 0.84% CDEA and 95.62% Glucidex IT-19.

### EXAMPLE 9

160 g of Empicol ESB 3 (27% active sodium laureth sulfate) were blended with 24 g of Amonyl 380 BA (30% active cocamidopropyl betaine) and 12 g Comperlan KD (cocamide DEA). 65 g of this solution was then poured on a blend of 80 g Laponite RD. The powder was then dried at 50° C. for 20 minutes. The dry composition was 14.23% SLES, 2.37% CAPB, 3.95% CDEA and 79.45% laponite RD.

In each of Examples 5 to 9, granules of mean particle diameter in the range 20 to 1000 μm were produced. The pH after wetting was estimated by dispersing 2 g granules in 200 g water and measuring pH of the resulting mixture, emulsion or dispersion. The softness to touch of the granules of each of Examples 5 to 9, both as produced and after rubbing with water on the hands, was regarded as attractive for a shampoo product, with the softness to touch of the Example 9 granules being particularly appreciated.

The products of each of Examples 5 to 9 were tested on hair.

The hair conditioning properties of the granulated shampoos are tested as follows: 1 g of each powder shampoo was applied on 10 g of wet black dyed hair tresses. The hair tresses were then rinsed with 200 g water and dried, and the ease of combing and softness to touch of the hair were assessed after drying on the following scale:
harsh and unmanageable
+ slightly harsh and/or difficult to comb
++ quite soft and combable
+++ soft and easily combed
++++ very soft and easily combed

The assessments for examples 5 to 9 are shown in Table 2.

TABLE 2

| | % dry active | | | | |
|---|---|---|---|---|---|
| Components | Example 5 | Example 6 | Example 7 | Example 8 | Example 9 |
| pH | 7.5 | 7 | 7 | 7 | 8 |
| Combing/ | +++ | +++ | ++ | +++ | +++ |
| softness | | | | | |

The powders of each of Examples 5 to 9 were packaged as 3 g powder in each of various paper packagings used commercially for other products and the packages were stored for 4 weeks at 35° C. and 70% humidity. The powder of Example 8 agglomerated under these conditions and was rated unsuitable for tropical climates. The condition of the other powders was assessed visually and by touch and rated as shown in Table 3:
Nice no visible agglomeration, powder retains its attractive soft touch
Agg some agglomeration visible and/or sensed by touch

If there was a residue on the packaging after storage, this is noted in Table 3 as y/p. The stored powders were tested on hair as described above, although the presence of a residue on the hair was assessed visually instead of being measured. None of the powders gave a visible residue after being rinsed. Those that showed a residue before rinsing which was removed after rinse are rated y/r in Table 3. Those that showed no residue are rated ‘No’.

TABLE 3

| Packaging | Example 5 | | | Example 6 | | | Example 7 | | | Example 9 | | |
|---|---|---|---|---|---|---|---|---|---|---|---|---|
| Soap | nice | +++ | y/r | nice | ++ | no | nice | ++ | no | nice | ++++ | y/r |
| paper | | | | | | | | | | | | |
| Sugar | nice | +++ | y/r | agg. | ++ | y/p | agg. | ++ | y/p | nice | ++++ | y/r |
| paper | | | | | | | | | | | | |
| Maizena | nice | +++ | y/r | nice | ++ | no | nice | ++ | no | nice | ++++ | y/r |
| paper | | | | | | | | | | | | |
| Bread- | nice | +++ | No | agg. | ++ | y/p | agg. | ++ | y/p | nice | ++++ | y/r |
| crumbs | | | | | | | | | | | | |
| paper | | | | | | | | | | | | |
| Flour | nice | +++ | y/r | nice | ++ | y/p | agg. | ++ | y/p | nice | ++++ | y/r |
| paper | | | | | | | | | | | | |
| Chicory | nice | +++ | y/r | nice | ++ | no | nice | ++ | no | nice | ++++ | y/r |
| paper | | | | | | | | | | | | |
| Uni-dose | nice | +++ | No | nice | ++ | no | agg. | ++ | no | nice | ++++ | y/r |
| sugar | | | | | | | | | | | | |
| paper | | | | | | | | | | | | |
| Glue paper | nice | +++ | y/r | nice | ++ | no | nice | ++ | no | nice | ++++ | y/r |
| Baby talc | nice | +++ | No | nice | ++ | no | nice | ++ | no | nice | ++++ | y/r |
| paper | | | | | | | | | | | | |
| Powder | nice | +++ | y/r | nice | ++ | no | nice | ++ | no | nice | ++++ | y/r |
| without | | | | | | | | | | | | |
| packaging | | | | | | | | | | | | |
| Powder | nice | +++ | y/r | nice | ++ | no | nice | ++ | no | nice | ++++ | y/r |
| not under | | | | | | | | | | | | |
| aging | | | | | | | | | | | | |

### EXAMPLE 10

160 g of Empicol ESB 3 (27% active sodium laureth sulfate) were blended with 24 g of Amonyl 380 BA (30% active cocamidopropyl betaine) and 12 g Comperlan KD (cocamide DEA). 2 g of an aqueous cationic emulsion of N-(aminoethyl)aminopropyl-substituted polydimethylsiloxane of viscosity 3500 cSt (DC 2-8299) were added to 49 g of surfactants solution. 35 g of this solution was then poured on 80 g native starch. The powder was then dried at 50° C. for 20 minutes. The dry composition was 8.1% SLES, 1.35% CAPB, 2.25% CDEA, 0.87% silicone and 87.43% starch.

### EXAMPLE 11

160 g of Empicol ESB 3 (27% active sodium laureth sulfate) were blended with 24 g of Amonyl 380 BA (30% active cocamidopropyl betaine) and 12 g Comperlan KD (cocamide DEA). 4 g of an aqueous cationic emulsion of N-(aminoethyl)aminopropyl-substituted hydroxy-terminated polydimethylsiloxane of viscosity 5 cSt (DC 949) were added to 49 g of surfactants solution. 40 g of this solution was then poured on 80 g native starch. The powder was then dried at 50° C. for 20 minutes. The dry composition was 8.78% SLES, 1.46% CAPB, 2.44% CDEA, 1.14% silicone and 86.18% starch.

### EXAMPLE 12

160 g of Empicol ESB 3 (27% active sodium laureth sulfate) were blended with 24 g of Amonyl 380 BA (30% active cocamidopropyl betaine) and 12 g Comperlan KD (cocamide DEA). 2.2 g of N-(aminoethyl)-2-methyl-3-aminopropyl-substituted polydimethylsiloxane fluid of viscosity 3500 cSt (DC 2-8566) were added to 49 g of surfactants solution. 40.5 g of this solution was then poured on 80 g native starch. The powder was then dried at 50° C. for 20 minutes. The dry composition was 9.08% SLES, 1.51% CAPB, 2.52% CDEA, 1.85% silicone and 85.03% starch.

### EXAMPLE 13

160 g of Empicol ESB 3 (27% active sodium laureth sulfate) were blended with 24 g of Amonyl 380 BA (30% active cocamidopropyl betaine) and 12 g Comperlan KD (cocamide DEA). 2 g of an aqueous nonionic emulsion of high viscosity polydimethylsiloxane (DC HV600) were added to 49 g of surfactants solution. 40 g of this solution was then poured on 80 g native starch. The powder was then dried at 50° C. for 20 minutes. The dry composition was 9.11% SLES, 1.52% CAPB, 2.53% CDEA, 0.84% silicone and 86% starch.

### EXAMPLE 14

160 g of Empicol ESB 3 (27% active sodium laureth sulfate) were blended with 24 g of Amonyl 380 BA (30% active cocamidopropyl betaine) and 12 g Comperlan KD (cocamide DEA). 2.16 g of an aqueous nonionic emulsion of high viscosity polydimethylsiloxane (DC HV600) were added to 49 g of surfactants solution. 44.5 g of this solution was then poured on a blend of 80 g native starch and 0.3 g deposition polymer Polyquat 10 Ucare JM 30M. The powder was then dried at 50° C. for 20 minutes. The dry composition was 9.91% SLES, 1.65% CAPB, 2.75% CDEA, 0.99% silicone, 0.3% Polyquat and 84.4% starch.

### EXAMPLE 15

160 g of Empicol ESB 3 (27% active sodium laureth sulfate) were blended with 24 g of Amonyl 380 BA (30% active cocamidopropyl betaine) and 12 g Comperlan KD (cocamide DEA). 2 g of an aqueous nonionic emulsion of a dimethylsiloxane diphenylsiloxane copolymer (DC 2-1388) were added to 49 g of surfactants solution. 40.2 g of this solution was then poured on 80 g native starch. The powder was then dried at 50° C. for 20 minutes. The dry composition was 9.13% SLES, 1.52% CAPB, 2.54% CDEA, 1.01% silicone and 85.8% starch.

### EXAMPLE 16

160 g of Empicol ESB 3 (27% active sodium laureth sulfate) were blended with 24 g of Amonyl 380 BA (30% active cocamidopropyl betaine) and 12 g Comperlan KD (cocamide DEA). 2 g of an aqueous cationic emulsion of N-(aminoethyl)aminopropyl-substituted polydimethylsiloxane of viscosity 3500 cSt (DC 2-8299) were added to 49 g of surfactants solution. 29.28 g of this solution was then poured on a blend of 40 g native starch and 40 g sodium sulfate. The powder was then dried at 50° C. for 20 minutes. The dry composition was 6.92% SLES, 1.15% CAPB, 1.92% CDEA, 0.74% silicone, 44.63% starch and 44.63% sodium sulfate.

### EXAMPLE 17

160 g of Empicol ESB 3 (27% active sodium laureth sulfate) were blended with 24 g of Amonyl 380 BA (30% active cocamidopropyl betaine) and 12 g Comperlan KD (cocamide DEA). 4 g of an aqueous cationic emulsion of N-(aminoethyl)aminopropyl-substituted hydroxy-terminated polydimethylsiloxane of viscosity 5 cSt (DC 949) were added to 49 g of surfactants solution. 31.5 g of this solution was then poured on a blend of 40 g native starch and 40 g sodium sulfate. The powder was then dried at 50° C. for 20 minutes. The dry composition was 7.12% SLES, 1.19% CAPB, 1.98% CDEA, 0.92% silicone, 44.39% starch and 44.39% sodium sulfate.

### EXAMPLE 18

160 g of Empicol ESB 3 (27% active sodium laureth sulfate) were blended with 24 g of Amonyl 380 BA (30% active cocamidopropyl betaine) and 12 g Comperlan KD (cocamide DEA). 2.2 g of N-(aminoethyl)-2-methyl-3-aminopropyl-substituted polydimethylsiloxane fluid of viscosity 3500 cSt (DC 2-8566) were added to 49 g of surfactants solution. 27.1 g of this solution was then poured on a blend of 40 g native starch and 40 g sodium sulfate. The powder was then dried at 50° C. for 20 minutes. The dry composition was 6.39% SLES, 1.07% CAPB, 1.78% CDEA, 1.3% silicone, 44.73% starch and 44.73% sodium sulfate.

The combing/softness was rated as:
+++ for examples 10, 11, 12, 13, 15, 17 and 18
++ for examples 14 and 16.

### EXAMPLES 19 TO 25

Granulated hair shampoo compositions were prepared by blending the sodium laureth sulphate with the silicone emulsion, pouring the mixture on a blend of native starch and sodium acetate and synthetic silicate. The mixture is stirred continuously until a particulate material is obtained. The particulate material is then passed over an Aeromatic spray granulator for 15 minutes at 55° C., generating the dry compositions described in Tables 4 and 5. Comparative liquid compositions were prepared by blending the liquid ingredients together in water such as to obtain the same active levels of silicone and sodium laureth sulphate.

The granulated hair shampoo compositions of Tables 4, 5A and 5B were applied to hair: a shampoo wash was carried out by applying about 1 g of each composition to 10 g of slightly bleached hair previously made wet (5 tresses of 2 g). The shampoo was worked into a lather and then rinsed out thoroughly with water. The initiation of foaming was very easy and the foam was airy. Panellists were asked to disentangle tresses while time was measured. The average recorded times and the standard deviations are given under the corresponding compositions. Static/fly away was measured on dry hair, as the angle obtained by combing each tress 3 times, the average angle and standard deviation are given under the corresponding compositions. Shine was assessed by comparing a tress treated with granulated shampoo composition vs a tress treated with liquid composition. Sensory evaluations were conducted via a triangular test where panellists had to find the different tress from the 2 others submitted.

TABLE 4

| | | | | | Example 21 | |
|---|---|---|---|---|---|---|
| | Example 19 | | Example 20 | | Dimethicone emulsion - | |
| | Dimethicone emulsion - | | Dimethicone emulsion - | | 500 000 cSt, with | |
| | 60 000 cSt | | 300 000 cSt | | cationic guar | |
| | | Comparative | | Comparative | | Comparative |
| Ingredients (% wt) | Granule | Liquid | Granule | Liquid | Granule | Liquid |
| Sodium laureth sulfate | 11.5 | 11.5 | 11.4 | 11.4 | 11.4 | 11.5 |
| Sodium acetate | 12.9 | | 12.9 | | 12.9 | |
| Synthetic silicate | 4.3 | | 4.3 | | 4.3 | |
| Starch | 68.8 | | 68.8 | | 68.7 | |
| Water | | 85.8 | | 86.0 | | 85.8 |
| Dimethicone Emulsion - 60 000 cSt | 2.5 | 2.7 | | | | |
| Dimethicone Emulsion - 300 000 cSt | | | 2.6 | 2.6 | | |
| Dimethicone Emulsion - 500 000 cSt, | | | | | 2.6 | 2.7 |
| with cationic guar | | | | | | |
| Wet Combing Time(s) | 16.9 ± 5.4 | 18.1 ± 8.6 | 10.4 ± 4.1 | 22.8 ± 11.6 | 16.3 ± 5.4 | 15.6 ± 6.7 |
| Static angle(°) | 19.7 ± 16.3 | 16.1 ± 4.8 | 9.5 ± 5.8 | 17.5 ± 6.2 | 15.5 ± 10.0 | 14.7 ± 6.7 |

TABLE 5A

| | Example 22 | | Example 23 | |
|---|---|---|---|---|
| | Dimethiconol | | Dimethiconol | |
| | emulsion (1) | | emulsion (2) | |
| | | Comparative | | Comparative |
| Ingredients (% wt) | Granule | Liquid | Granule | Liquid |
| Sodium laureth | 11.5 | 11.5 | 11.4 | 11.5 |
| sulfate | | | | |
| Sodium acetate | 12.9 | | 12.9 | |
| Synthetic silicate | 4.3 | | 4.3 | |
| Starch | 68.8 | | 68.8 | |
| Water | | 85.9 | | 85.9 |
| Dimethiconol | 2.5 | 2.6 | | |
| emulsion (1) | | | | |
| Dimethiconol | | | 2.7 | 2.6 |
| emulsion (2) | | | | |
| Dimethiconol | | | | |
| emulsion (3) | | | | |
| Bis (C13-15 Alkoxy) | | | | |
| PG-Amodimethicone | | | | |
| Cocamidopropyl | | | | |
| betaine | | | | |
| Cocamide DEA | | | | |
| Wet Combing | 19.8 ± 6.4 | 22.5 ± 7.5 | 18.5 ± 6.3 | 15.2 ± 5.8 |
| Time (s) | | | | |
| Static angle (°) | 21.6 ± 9.8 | 17.4 ± 4.3 | 16.4 ± 7.2 | 19.2 ± 3.8 |

TABLE 5B

| | Example 24 | | Example 25 | |
|---|---|---|---|---|
| | Dimethiconol | | Bis (C13-15 Alkoxy) | |
| | emulsion (3) | | PG-Amodimethicone | |
| | | Comparative | | Comparative |
| Ingredients (% wt) | Granule | Liquid | Granule | Liquid |
| Sodium laureth | 11.4 | 11.4 | 8.0 | 8.0 |
| sulfate | | | | |
| Sodium acetate | 12.9 | | 12.9 | |
| Synthetic silicate | 4.3 | | 4.3 | |
| Starch | 68.8 | | 68.8 | |
| Water | | 86.1 | | 86.3 |
| Dimethiconol | | | | |
| emulsion (1) | | | | |
| Dimethiconol | | | | |
| emulsion (2) | | | | |
| Dimethiconol | 2.5 | 2.5 | | |
| emulsion (3) | | | | |
| Bis (C13-15 Alkoxy) | | | 2.6 | 2.2 |
| PG-Amodimethicone | | | | |
| Cocamidopropyl | | | 1.3 | 1.3 |
| betaine | | | | |
| Cocamide DEA | | | 2.2 | 2.2 |
| Wet Combing | 22.9 ± 4.9 | 14.3 ± 5.5 | 23.1 ± 6.3 | 18.9 ± 7.1 |
| Time (s) | | | | |
| Static angle (°) | 12.8 ± 3.9 | 18.5 ± 4.1 | 1.6 ± 5.3 | 13.4 ± 4.4 |

Results indicated that the various granulated shampoo compositions and corresponding liquids are equivalent for most of the parameters: detangling time, static angle and shine. For Example 20, the hair treated with the granulated shampoo composition was easier to comb and less static than the hair treated with the corresponding liquid, while for Example 24 it was the reverse. There was mainly no observed difference for the shine between hair treated with the granulated shampoo compositions and hair treated with the corresponding liquids. There was generally a smoother feel for the liquid version compared to the granulated version.

### EXAMPLES 26 AND 27

Solubility of granulated shampoo compositions may be fine tuned depending on the type of surfactants used in the mixture of the liquid feed. The dry compositions of Example 26 and Example 27, described in Table 6, were prepared as follows: the dimethiconol emulsion was mixed with the surfactant or the mixture of surfactants, until a homogeneous solution was obtained. The solution thus prepared was poured into a high shear mixer in which corn starch, sodium acetate and synthetic silicate were placed. The mixture was stirred continuously until a particulate material was obtained. The particulate material was then passed over an Aeromatic spray granulator for 15 minutes at 55° C. Example 27 containing cocamidopropyl betaine and cocamide DEA in addition to the sodium laureth sulfate was found easier to solubilize in water upon application on hair compared to Example 26 which only contains sodium laureth sulfate.

TABLE 6

| Ingredients (% wt) | Example 26 | Example 27 |
|---|---|---|
| Sodium laureth sulfate | 11.40 | 7.98 |
| Cocamidopropyl betaine | | 1.30 |
| Cocamide DEA | | 2.17 |
| Dimethiconol emulsion | 2.54 | 2.54 |
| Synthetic silicate | 4.30 | 4.30 |
| Sodium acetate | 12.91 | 12.90 |
| Corn starch | 68.85 | 68.81 |

### EXAMPLES 28 TO 30

Granulated shower gel compositions were prepared by blending the sodium laureth sulphate, decyl glucoside, cocamidopropyl betaine and laureth-4 with the silicone emulsion or fluid, pouring the mixture on a blend of native starch and sodium acetate. The mixture was stirred continuously until a particulate material was obtained. The particulate material was then passed over an Aeromatic spray granulator for 15 minutes at 55° C., generating the dry compositions described in Table 7, for Examples 28 to 30. The obtained granulated shower gel compositions were compared to each other by 4 panellists. Panellist's comments confirmed the softness and ease of use of the granulated shower gels in terms of dissolution upon use, foaming, airy and rich quality of foam, ease of rinse, smoothness and suppleness of skin after drying.

TABLE 7

| Ingredients (% wt) | Example 28 | Example 29 | Example 30 |
|---|---|---|---|
| Sodium laureth sulfate | 6.4 | 6.3 | 6.3 |
| Decyl glucoside | 2.1 | 2.1 | 2.1 |
| Cocamidopropyl betaine | 2.3 | 2.2 | 2.5 |
| Laureth-4 | 1.5 | 1.5 | 1.5 |
| Bis(C13-15 Alkoxy) | 1.7 | | |
| PG-Amodimethicone | | | |
| Divinyldimethicone/ | | 1.6 | |
| Dimethicone | | | |
| Copolymer emulsion | | | |
| Bis-PEG-18 Methyl Ether | | | 1.9 |
| Dimethyl Silane | | | |
| Starch | 73.1 | 73.3 | 72.9 |
| sodium acetate | 12.9 | 12.9 | 12.9 |

### EXAMPLE 31

A granulated hair shampoo composition was prepared by blending the sodium laureth sulphate with the silicone emulsion, pouring the mixture on a blend of sodium acetate, synthetic silicate and synthetic calcium silicate. The mixture was stirred continuously until a particulate material was obtained. The particulate material was then passed over an Aeromatic spray granulator for 15 minutes at 55° C., generating the dry composition described in Table 8.

TABLE 8

| Ingredients (% wt) | Example 31 |
|---|---|
| Sodium laureth sulfate | 40.38 |
| Dimethicone emulsion-500 000 cSt, | 9.22 |
| with cationic guar | |
| Sodium acetate | 7.63 |
| Synthetic silicate | 2.54 |
| Synthetic calcium silicate | 40.23 |

### COMPARATIVE EXAMPLES 1 TO 4

Comparative examples 1 to 4 were formulated using different ingredients such as powder sodium lauryl sulphate in powder form and high amylose corn starch, using the granulation technique, instead of the extrusion technique such as described in US2004/0202632. Sodium lauryl sulphate, cocamidopropylbetaine and cocamide DEA are heated at 65° C. until a homogeneous solution was obtained. The dimethicone copolyol emulsion was added to this mix under agitation. The solution thus prepared was poured into a high shear mixer in which the carrier powders were placed. The mixture was stirred continuously until a particulate material was obtained. The particulate material was then passed over an Aeromatic spray granulator for 5 minutes at 45° C. The dry compositions of the comparative examples were described in Table 8. The obtained compositions were dusty powders with unpleasant feel and presence of hard waxy agglomerates, which did not resemble the granulated powders obtained when working with liquid surfactants and natural starch.

TABLE 8

| Ingredients | Comparative | Comparative | Comparative | Comparative |
|---|---|---|---|---|
| (% wt) | example 1 | example 2 | example 3 | example 4 |
| Sodium lauryl | 10.64 | 10.51 | 10.54 | 10.47 |
| sulphate | | | | |
| Cocamido- | 1.60 | 1.58 | 1.58 | 1.57 |
| propyl betaine | | | | |
| Cocamide DEA | 2.66 | 2.63 | 2.63 | 2.62 |
| Dimethicone | 0.56 | 0.56 | 0.56 | 0.55 |
| copolyol | | | | |
| emulsion | | | | |
| High amylose | 84.55 | | 67.75 | |
| corn starch | | | | |
| Natural | | 84.72 | | 67.40 |
| corn starch | | | | |
| Synthetic | | | 4.23 | 4.35 |
| silicate | | | | |
| Sodium acetate | | | 12.70 | 13.04 |

It was demonstrated that shampoo can be formulated in powder form in the presence of a carrier. These formulations exhibit a pleasant feel on the skin before and after applying in the presence of water. These benefits are kept after aging in paper-based packaging.

1. A granulated personal care shampoo comprising a shampoo composition comprising at least one surfactant agglomerated with a water-soluble, water-dispersible or water-insoluble solid particulate carrier.

2. The granulated shampoo according to claim 1, characterized in that the shampoo composition additionally contains a conditioner preferably comprising an organopolysiloxane.

3. The granulated personal care shampoo according to claim 1, characterized in that the carrier is water-soluble and comprises sodium sulphate, maltodextrin or dextrose.

4. The granulated personal care shampoo according to claim 1, characterized in that the carrier comprises a water-dispersible clay or starch.

5. The granulated personal care shampoo according to claim 1, characterized in that the mean particle diameter of the granules is in the range 0.02 to 1.5 mm, preferably 0.02-1 mm, more preferably 0.05-0.8 mm, even more preferably 0.1-0.6 mm.

6. The granulated personal care shampoo according to claim 1, characterized in that the weight ratio of dry shampoo composition to carrier is in the range 2:98 to 40:60.

7. The granulated personal care shampoo according to claim 1, characterized in that the shampoo composition contains a binder.

8. A process for the preparation of a shampoo in powder form, characterized in that a liquid shampoo composition comprising at least one surfactant which has been solubilised in water or molten is contacted with a solid particulate carrier under conditions such that the surfactant is agglomerated with the carrier, the agglomerated product being kept in granule form during agglomeration or subsequently formed into granules.

9. The process according to claim 8, characterized in that the carrier is water-soluble or water-dispersible and, preferably, the mean particle diameter of the carrier fed to the mixer is between 1 micrometer and 250 micrometer.

10. The process according to claim 8, characterized in that the carrier is water-insoluble and the mean particle diameter of the carrier fed to the mixer is between 1 micrometer and 10 micrometer.

11. The process according to claims 8, characterized in that the surfactant is contacted with the carrier in a granulating mixer in which the agglomerated product is kept in granule form.

12. The process according to claim 8, characterized in that the liquid shampoo composition is sprayed onto the carrier.

13. The process according to claim 11, characterized in that the mixer is a vertical continuous granulating mixer comprising blades rotating within a tubular housing and having an inlet for solid carrier particles and a spray inlet for the liquid shampoo composition to contact the solid particles above the blades.

14. The process according to claim 8, characterized in that the ratio of the weight of shampoo composition to the weight of carrier after drying is in the range 2:98 to 40:60.

15. A package comprising an envelope which is at least partially biodegradable containing granules of the personal care shampoo according to any preceding claim.

16. A shampoo for human or animal hair comprising granules obtained from the process according to claim 8.

17. A personal care product for keratinous membranes comprising granules obtained from the process according to claim 8.
